(19) United States
(12) Patent Application Publication
(10) Pub. No.: US 2011/0305779 A1
(43) Pub. Date: Dec. 15, 2011

(54) PHYTOCHEMICAL COMBINATIONS THAT REGULATE PATHOLOGICAL IMMUNITY

(76) Inventor: Fred M. COWAN, Colora, MD (US)

(21) Appl. No.: 13/157,040

(22) Filed: Jun. 9, 2011

Related U.S. Application Data
(60) Provisional application No. 61/353,526, filed on Jun. 10, 2010.

(51) Int. Cl. A61K 36/9068; A61K 36/81; A61P 29/00; A61K 36/9066

(52) U.S. Cl. 424/739; 424/756; 424/760

(57) ABSTRACT

Disclosed are dietary supplements comprising a combination of a spice, a partially purified phytochemical, and a fully purified phytochemical that synergistically downregulates inflammation and promotes wellness, good health, and fitness in a mammal. Methods of identifying and making such supplements are likewise provided. The method of identifying comprises contacting mammalian cells, in vitro or in vivo, with the combination, assaying to detect a change in the level of a biomarker that is correlated with downregulation of an inflammatory response of the cells, and identifying a phytochemical composition comprising a combination of spices that synergistically changes the level of the biomarker. Exemplary supplements include a combination of spices selected from (a) turmeric and ginger, (b) turmeric and capsicum, and (c) paprika and nutmeg.

### CROSS-REFERENCE TO RELATED APPLICATIONS

This application relies on the disclosure of, and claims the benefit of the filing date of, U.S. provisional patent application No. 61/353,526, filed Jun. 10, 2010, the entire disclosure of which is hereby incorporated herein by reference.

### BACKGROUND OF THE INVENTION

1. Field of the Invention

The present invention relates generally to the field of making and administering to a subject phytochemical formulations that downregulate pathological inflammation in a subject in need of such treatment. More specifically, the present invention relates to making and administering dietary supplements comprising phytochemical formulations containing two or more spices, fully or partially purified phytochemicals, or a combination of these, that synergistically downregulate pathological inflammation, to promote wellness, good health, and fitness, and to prevent or reduce undesirable effects of pathological inflammation, in a subject in need of such treatment. Such undesirable effects of inflammation include but are not limited to tissue-damaging inflammatory responses associated with cardiovascular disease, diabetes, hay fever and other allergies, asthma, Alzheimer's disease, anemia, rheumatoid arthritis and other autoimmune diseases, and cancer. Such undesirable effects also include undesirable actions on other immune processes that are influenced or regulated by inflammatory responses that are downregulated by the present invention. The present invention further relates to in vitro and in vivo assays that identify such combinations of phytochemicals that synergistically inhibit pathological inflammation, as evidenced by their ability to synergistically alter the level of a biomarker that is correlated with downregulation of an inflammatory response.

2. Description of the Related Art

Phytochemical synergy is relevant to diet, dietary supplements, and drug discovery, crossing the boundaries between commercial food, dietary supplement, and pharmaceutical interests. Synergy in the metabolic or physiological activities of a combination of phytochemicals is a recurrent theme in many processes contributing to good nutrition, traditional herbal medicine, dietary supplements, and pharmaceuticals. However, while activities of individual phytochemicals have received considerable attention and investigation, synergistic physiological or pharmacological activities of phytochemical combinations in general, and phytochemical combinations that act synergistically to inhibit pathological inflammation in particular, have received little study and are poorly utilized.

A major limitation on the use of phytochemicals in healthcare is that most of these compounds individually have relatively low potencies and so require high doses to achieve even modest efficacy. That limitation might be overcome by use of formulations more reflective of a healthy diet containing multiple phytochemicals that interact collectively and synergistically. This phenomenon, called “phytochemical synergy” is a major focus of the current application.

The physiological and pharmacological mechanisms by which phytochemicals affect pro-inflammatory signaling pathways are being considered at the cutting-edge of contemporary nutritional science. Spice-derived phytochemicals have been shown to inhibit both redox reactions and signaling mechanisms such as the nuclear factor-kappaB (NF-kB) pathway, offering promise for the management of pathological inflammation (Aggarwal and Shishodia 2004). Processes that involve NF-kB have emerged as important mediators of disease- or trauma-associated inflammation, particularly those with increased incidence among elderly populations such as atherosclerosis and other cardiovascular disorders, diabetes, allergies, asthma, Alzheimer's disease, osteoporosis, cancer, and many forms of autoimmunity (Aggarwal and Shishodia 2004). While the focus of the present invention is on downregulating inflammation and alleviating disease, insult and injury associated therewith, the actions of phytochemicals are not limited to purely inflammatory responses and can also influence other metabolic and physiological processes, including other immune responses.

An example of research that may foster increasing use of phytochemicals in preventive medicine and therapy is provided by characterization of SIRT1, a histone deacetylase (HDAC) sirtuin enzyme upregulated by both stress and the grape phytochemical resveratrol. SIRT1 directly binds to and regulates molecules of the NF-kB cascade (Yeung et al., 2004), and is also involved in epigenomic regulation of gene expression (Aggarwal & Sishodia 2004; Chung et al., 2009; Kawahara et al., 2009). The SIRT1 sirtuin enzyme physically interacts with the NF-kB RelA/p65 subunit, to suppress NF-kB-mediated gene expression (Yeung et al., 2004). One specific outcome of this activity is that by acting through SIRT1 to inhibit NF-kB, resveratrol can enhance TNF-mediated apoptosis (Yeung et al., 2004). Apoptosis of inflammatory cells, in particular neutrophils, is a major mechanism for down-regulating inflammation and preventing it from becoming pathological (reviewed in Martin, 2006).

Resveratrol is a polyphenol phytochemical found in the skin of red grapes. It has diverse physiological properties, including estrogenic, antiplatelet, and anti-inflammatory activities. Resveratrol is reported to act as an anti-aging compound, and to confer health benefits ranging from chemoprevention to cardioprotection (reviewed by Das et al., 2010). Resveratrol and its analogs are a current focus of drug discovery efforts. Resveratrol analogs that modulate expression of sirtuins are being developed as proprietary small molecule drugs with the potential to treat age-associated diseases such as type 2 diabetes (see the publications listed on the website of Sirtris Pharmaceuticals, sirtrispharma.com, last visited on May 28, 2010).

Curcumin, the major active phytochemical component of the spice turmeric, has shown some efficacy against cancer when administered in high doses (Aggarwal et al. 2003). Even growth of pancreatic cancer, which is notoriously resistant to therapy, can be inhibited in some patients by oral administration of curcumin (Dhillon et al., 2008). Curcumin is the subject of research to develop more potent analogs with increased bioavailability (Aggarwal and Shishodia 2004).

Capsaicin (trans-8-methyl-N-vanillyl-6-nonenamide) is the principal “hot” ingredient present in the seeds and stem of peppers of the genus Capsicum (Solanaceae). Capsaicin is reported to block the translocation of nuclear factor kappa B (NF-kB), activator protein 1 (AP-1), and signal transducer and activator of transcription (STAT3) signaling pathway that are required for carcinogenesis, and to have cancer-suppressing activity (e.g., see Aggarwal and Shishodia 2004). Capsaicin is also reported to have anti-inflammatory activity attributed to an inhibitory effect on cellular COX-2 activity (Oyagbemi et al., 2010). Capsaicin has analgesic (pain-reducing) activity and is used as both a topical and an injectable analgesic. It is not an FDA-approved product but is currently in Phase 3 trials for postoperative pain control, arthritis, musculoskeletal pain, and chronic neuropathic pain. (Vadivelu et al., 2010).

The operation of phytochemical synergy in combating cancer is described by Morré and Morré, 2003. A 25:1 mixture of decaffeinated green tea concentrate and a preparation of capsicum from African bird peppers (Capsicum frutescens) is shown to exhibit synergistic anti-cancer properties. The combination of catechin in green tea and the vanilloid capsaicin is reported to inhibit tumor-associated NADH oxidase (tNOX), which blocks cancer cell enlargement after cell division, and kills the cancer cells (Morré and Morré, 2003).

McCarty (2004) suggested using multiple phytochemicals to target multiple signaling pathways as a strategy for managing cancer, an approach they termed “Multifocal Signal Modulation Therapy.”

Other investigators have demonstrated that a combination of curcumin and resveratrol exhibits 4-fold antioxidant synergism in heme-enhanced oxidation reactions (Aftab and Vieira 2009).

A promising area of research that is beginning to receive attention of researchers is the ability of phytochemicals to augment the therapeutic potential of pharmaceutical drugs by enhancing their potency, which allows a reduction in drug dose (and drug-associated toxicity) (Haines et al., 2000; Martin 2006; Cowan et al., 2006). An example of this effect is provided by use of Ginkgo biloba extracts to lower the dosage of the immunosuppressive drug, FK506 for cardioprotection in vivo (Haines et al., 2000). Related studies show that ginkgolide B, a component molecule of Ginkgo biloba, reduces the dosage of the immunosuppressant drug cyclosporin A required to inhibit inflammatory cytokine expression by human T cells in vitro (Mahmoud et al., 2004). Ginkgo leaf extract has also been shown to interact with vitamin C and the antioxidant carotenoid astaxanthin to mediate anti-inflammatory effects with the same potency as ibuprofen, but without risk of adverse gastrointestinal side effects caused by ibuprofen and other NSAIDs (Haines et al 2010). Studies such as these demonstrate that inflammation and T cell-mediated immunity are not segregated, but are conjoined, integrated immune regulatory processes. The actions of phytochemicals on inflammation occur in concert with their effects on T cell-mediated immunity. Accordingly, the biomarkers used to indentify phytochemical synergy are indicative of these integrated immune processes.

Examples of modulation of biochemical, physiological and pharmacological mechanisms by phytochemicals have been reviewed; for example, links between NF-kB inflammatory pathways and the NF-kB inhibitory and anti-inflammatory properties of phytochemicals derived from spices are described in Aggarwal and Shishodia (2004).

Martin (2006) has written a comprehensive review of the effects of dietary constituents, particularly phytochemicals, on the complex process of apoptosis, a form of programmed cell death. The review describes apoptotic action induced by NF-kB pathway-inhibitory phytochemicals as a pivotal defense against pathological inflammation and disease.

Interleukin (IL)-8 is a cytokine produced at the end of NF-kB mediated cytokine cascades, and the level of IL-8 in peripheral blood is a reliable biomarker of pathological inflammation and efficacy of anti-inflammatory therapies. IL-8 is chemotactic, strongly recruiting neutrophils and inhibiting their apoptosis, thereby impairing resolution of inflammation and establishing conditions for chronic disease (Aggarwal & Sishodia 2004; Martin 2006). In previous studies elevated levels of IL-8 have been associated with insult, injury and a variety of pathologies and disease; e.g., chemical insult, specifically sulfur mustard exposure, and acute respiratory distress syndrome(ARDS) and associated multi-organ dysfunction syndrome (MODS) (reviewed in Cowan et al., 2004; and Surbatovic et al., 2007). Sulfur mustard increased-IL-8 is a biomarker for drug and phytochemical efficacy against this chemical agent (reviewed in Cowan et al., 2004; 2006); and along with CRP and the pro-inflammatory cytokine IL-6, is an indicator of pulmonary disease severity in mustard-exposed persons (Attaran et al., 2009; Pourfarzam et al., 2009).

The phytochemical capsaicin and its more potent drug analogues inhibit sulfur mustard-increased IL-8 production in cell culture models, and show efficacy against sulfur mustard pathology in the mouse vesicant model (reviewed Cowan et al., 2004, 2006). Phytochemicals such as capsicum, curcumin, and resveratrol are able to inhibit IL-8 production and reverse the anti-apoptotic and pro-inflammatory effects of this cytokine (reviewed, Aggarwal and Shishodia 2004; Martin 2006). Such apoptotic pharmacology is also observed for phytochemicals and drugs used as chemical agent countermeasures (reviewed, Cowan et al., 2004, 2006; Martin 2006).

Foods contain complex mixtures of phytochemicals that are capable of influencing biomarkers associated with immunoregulation. Examples include cruciferous vegetables such as broccoli, which are rich in isothiocyanate phytochemicals such as sulforaphane. A human clinical study revealed that pro-inflammatory HDAC activity in PBMCs of healthy individuals was significantly reduced 3-6 hours following consumption of broccoli sprouts (Myzak et al., 2007). Thus consumption of even a small serving of a single food item can influence an immunoregulatory biomarker in human subjects, validating biomarkers as a measure of phytonutrition. This might be relevant to the direct signaling transduction relationship that exists between the p65 (Re1A) subunit of NF-kB, which interacts with the HDAC co-repressors HDAC1 and HDAC2 to regulate NF-kB-mediated gene expression (Ashburner et al., 2001).

Epidemiological studies show a clear correlation between a diet rich in fruits and vegetables and a reduced risk of chronic diseases such as cancer and cardiovascular disease. As with overall nutrition, studies of the antioxidant components in fruit and vegetable phytochemical extracts show that the benefit of a diet rich in fruits and vegetables is attributed to the complex mixture of phytochemicals present in whole foods, and that no single antioxidant can replace the combination of natural phytochemicals in fruits and vegetables to achieve the health benefits (Liu 2004). Published literature in the field has recognized the benefits of a diet containing many different phytochemicals; however, it does not describe or suggest the methods of the present invention for identifying phytochemical combinations that synergistically inhibit pathological inflammatory responses in mammals. Nor does the literature describe or suggest using such methods to make and orally administer prophylactic and therapeutic compositions of the present invention containing combinations of phytochemicals that synergistically inhibit inflammation, to promote wellness, good health and fitness, and to prevent or reduce the effects of inflammation in a mammalian subject in need of such treatment.

### BRIEF SUMMARY OF THE INVENTION

Dysregulated, chronic inflammation is a common pathological condition that underlies and/or aggravates almost all common diseases and disorders, including cardiovascular disease, allergies, asthma, rheumatoid arthritis, osteoarthritis, Alzheimer's disease, cancers, and autoimmune diseases in general. It is currently treated with proprietary small molecule drugs that act at single critical checkpoints in the inflammatory cascade and effectively suppress major symptoms of many diseases. Such proprietary small molecule drugs are typically costly, toxic, and not effectively used prophylactically. Many phytochemicals widely distributed in human diet, particularly in common spices, have anti-inflammatory properties, but individually provide little or no therapeutic or prophylactic benefit.

The present invention provides methods and compositions that use phytochemical “blends” to synergistically downregulate, i.e., suppress or inhibit, pathological inflammation, which is involved in insult and injury, and is known to contribute to pathological syndromes and diseases in humans and other mammals. An object of the invention is to provide methods comprising orally administering to a human or other mammalian subject a physiologically or pharmacologically effective dose of such a synergistically acting phytochemical blend, to promote such things as wellness, good health, and fitness. The invention provides methods that comprise orally administering a physiologically or pharmacologically effective dose of a synergistically acting phytochemical blend to a human or other mammalian subject to alleviate or reduce harmful effects of pathological inflammation in a subject in need of such treatment. The invention also provides methods comprising orally administering a physiologically or pharmacologically effective dose of a synergistically acting phytochemical blend to a human or other mammalian subject to prevent harmful effects of pathological inflammation in a subject in need of such treatment. In the aforementioned methods of the invention, the synergistically-acting phytochemical blend can be a dietary supplement or seasoning comprising fully or partially purified phytochemicals, phytochemical-containing spices and/or herbs, or extracts thereof, or a combination of these, that synergistically downregulate inflammation in a mammal, which phytochemical blend can further be fortified with one or more fully or partially purified phytochemical(s) or drug(s) to increase potency and achieve efficacy.

An object of the present invention is to provide a method for identifying a phytochemical composition comprising two or phytochemical constituents that synergistically downregulate inflammation in a mammal, comprising: (i) incubating mammalian cells in vitro in solution containing a phytochemical composition comprising two or more phytochemical constituents selected from the group consisting of a spice, a partially purified phytochemical, and a fully purified phytochemical; (ii) assaying to detect a change in the level of a biomarker, which change is correlated with downregulation of an inflammatory response of the cells; and (iii) identifying a phytochemical composition comprising two or more phytochemical constituents that synergistically effect a change in the level of the biomarker in a manner that is correlated with downregulation of an inflammatory response of the cells.

The invention can be practiced using any mammalian cells that express or produce a biomarker at a level that is correlated with the level of an inflammatory response of the cells under the conditions of the assay. For example, in one embodiment of the invention, the cells are human peripheral blood mononuclear cells (PBMCs).

In practicing the methods of the invention described herein, the biomarker can be selected from the group consisting of IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), and histone deacetylase (HDAC).

In order to demonstrate the successful operation of the invention, without being limited thereby, examples of the method of the invention are described herein in which the cells are human PBMCs and the biomarker is IL-8. The examples described herein demonstrate successful use of the claimed method to identify three different combinations of spices that synergistically downregulate a mammalian inflammatory response: (a) turmeric and ginger, (b) turmeric and capsicum, and (c) paprika and nutmeg (see Examples 1-3).

Accordingly, the present invention includes the above-described method for identifying a phytochemical composition comprising two or more phytochemical constituents that synergistically downregulate inflammation in a mammal, wherein the phytochemical composition of step (i) comprises the two spices selected from the group consisting of (a) turmeric and ginger, (b) turmeric and capsicum, and (c) paprika and nutmeg, in amounts that synergistically effect a change in the level of the biomarker in a manner that is correlated with downregulation of an inflammatory response of the cells.

It is also an object of the present invention to provide a method for making a dietary supplement comprising a phytochemical composition that downregulates inflammation in a mammal, comprising: preparing a dietary supplement comprising a phytochemical composition comprising a mixture of two or more phytochemical constituents selected from the group consisting of a spice, a partially purified phytochemical, and a fully purified phytochemical, which combination of phytochemical constituents synergistically changes the level of a biomarker of mammalian cells in vitro in a manner that is correlated with downregulation of an inflammatory response of the cells, wherein said two or more phytochemical constituents are present in amounts effective to synergistically downregulate inflammation in a mammal.

Another object of the present invention is to provide a method for identifying a phytochemical composition comprising two or more phytochemical constituents that synergistically downregulate inflammation in a mammal, comprising: (i) orally administering to a mammal a phytochemical composition comprising two or more phytochemical constituents selected from the group consisting of a spice, a partially purified phytochemical, and a fully purified phytochemical, (ii) assaying to detect a change in the level of a biomarker, which change is correlated with downregulation of inflammation in the mammal following administration of the phytochemical composition, and (iii) identifying a phytochemical composition comprising two or more phytochemical constituents that synergistically effect a change in the level of the biomarker in a manner that is correlated with downregulation of inflammation in the mammal.

It is also an object of the present invention to provide a method for making a dietary supplement comprising a phytochemical composition that downregulates inflammation in a mammal, comprising: preparing a dietary supplement comprising a phytochemical composition comprising a mixture of two or more phytochemical constituents selected from the group consisting of a spice, a partially purified phytochemical, and a fully purified phytochemical, which combination of phytochemical constituents synergistically effects a change in the level of a mammalian biomarker in a manner that is correlated with downregulation of inflammation in a mammal following oral administration of the phytochemical composition, wherein said two or more phytochemical constituents are present in the dietary supplement in amounts effective to downregulate inflammation in a mammal.

The present invention includes embodiments of the above-described method for making a dietary supplement, wherein the dietary supplement comprises a phytochemical composition comprising two spices selected from the group consisting of (a) turmeric and ginger, (b) turmeric and capsicum, and (c) paprika and nutmeg, in amounts effective to downregulate inflammation in a mammal following oral administration of the phytochemical composition.

Once a phytochemical composition comprising two or more phytochemical constituents (purified phytochemicals, spices, or a combination thereof) that synergistically downregulate mammalian inflammation is identified, it is an object of the invention to further provide methods that can be used to identify and prepare an optimized phytochemical composition containing a ratio of the synergistically acting constituents that is selected to provide a relatively high degree of synergistic downregulation of inflammation in a mammal.

The invention provides in vitro methods for identifying an optimized phytochemical composition comprising two or more phytochemical constituents that are present in a ratio selected to provide a desired degree of synergistic downregulation of an inflammatory response of mammalian cells, the methods comprising: (i) preparing multiple phytochemical compositions comprising various selected ratios of two or more phytochemical constituents selected from the group consisting of a spice, a partially purified phytochemical, and a fully purified phytochemical, (ii) incubating mammalian cells in vitro in solutions, each of which contains one of the multiple phytochemical compositions of step (i), (iii) assaying each cell sample to detect a change in the level of a biomarker, which change is correlated with downregulation of an inflammatory response of the cells, and (iv) identifying a phytochemical composition comprising two or more phytochemical constituents that synergistically downregulate an inflammatory response of mammalian cells, which constituents are present in a ratio that provides a desired level of relative synergistic activity.

In practicing the above-described in vitro method of the invention, the biomarker can be, but is not necessarily, selected from the group consisting of IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), and histone deacetylase (HDAC). In addition, other known biomarkers, the levels of which are known to correlate with the level of an inflammatory response of mammalian cells, can also be used effectively.

The invention also provides in vivo methods for identifying an optimized phytochemical composition comprising two or more phytochemical constituents that are present in a ratio selected to provide a desired degree of synergistic downregulation of inflammation in a mammal, the methods comprising: (i) preparing multiple phytochemical compositions comprising various selected ratios of two or more phytochemical constituents selected from the group consisting of a spice, a partially purified phytochemical, and a fully purified phytochemical, (ii) orally administering each of the multiple phytochemical compositions to a mammalian subject, and assaying to detect a change in the level of a biomarker, which change is correlated with downregulation of inflammation in the mammal following administration of the phytochemical composition, and (iii) identifying a phytochemical composition comprising two or more phytochemical constituents that synergistically downregulate inflammation in a mammal, which constituents are present in a ratio that provides a desired level of relative synergistic activity.

All of the above-described methods of the invention that comprise orally administering a phytochemical composition to a mammal and assaying to detect a change in the level of a biomarker that is correlated with synergistic downregulation of inflammation in the mammal can be practiced effectively using a biomarker selected from the group consisting of C-reactive protein (CRP), IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), histone deacetylase (HDAC), intercellular adhesion molecule 1 (ICAM 1), myeloperoxidase (MPO), and soluble CD40 ligand (sCD40L).

All of the above-described in vitro and in vivo methods for identifying a combination of phytochemical constituents that synergistically downregulates a mammalian inflammatory response, comprising assaying to detect a change in the level of a biomarker, can be practiced effectively using assay methodologies that qualitatively determine the effects of the phytochemical constituents on the level of the biomarker; e.g., by detecting a “strong” change vs. a “weak” change or “little or no” change in the level of the biomarker. Alternatively, all of the identification methods of the invention can be practiced effectively using assay methodologies that semi-quantitatively determine the effects of the phytochemical constituents on the level of the biomarker; e.g., by detecting reduction to a level of “about two-thirds” or “about three-quarters” of the control level. All of the methods of the invention for identifying a synergistically acting combination of phytochemical constituents can also be practiced effectively using assay methodologies that quantitatively determine the effects of the phytochemical constituents on the level of the biomarker; e.g., by sensitively detecting changes in the level of the biomarker with relatively high accuracy and precision, e.g., to within 1-10%, 1-5%, or 1-3% of the actual value.

The methods, dietary supplements, and other orally administered compositions of the present invention that use or comprise a phytochemical composition comprising a mixture of two or more phytochemical constituents selected from the group consisting of a spice, a partially purified phytochemical, and a fully purified phytochemical, operate effectively in embodiments wherein the synergistically acting phytochemical constituents all have the same general form; e.g., all are spices, or all are partially purified phytochemicals. Likewise, the methods, dietary supplements, and other orally administered compositions of the present invention also operate effectively in embodiments wherein the phytochemical composition of the invention comprises a combination of synergistically acting phytochemical constituents having different forms, e.g., a spice and a fully purified phytochemical, or two spices and a partially purified phytochemical.

As described above, the invention provides methods that can be used effectively to identify phytochemical compositions that synergistically downregulate inflammation in a mammal. The invention provides methods for preparing dietary supplements and other orally administered compositions containing such phytochemical compositions that reduce or alleviate harmful effects of pathological inflammation in a mammalian subject. The invention also provides methods for preparing dietary supplements and other orally administered compositions containing such phytochemical compositions that prevent harmful effects of pathological inflammation in a mammalian subject. In addition, the invention provides methods for preparing dietary supplements and other orally administered compositions containing such phytochemical compositions that can be administered to a mammalian subject to effectively promote such things as wellness, good health, or fitness in the subject. Examples of such dietary supplements and other orally administered compositions of the invention that are suitable for reducing or alleviating harmful effects of pathological inflammation, for preventing harmful effects of pathological inflammation, or for promoting wellness, good health, and fitness, in a mammalian subject, are described below.

An object of the invention is to provide a dietary supplement comprising a phytochemical composition comprising 5% to 70% turmeric, 3% to 50% ginger, 0.5% to 50% cinnamon, and 0.05% to 30% capsicum. Of course, those of skill in the art will immediately recognize that each individual value falling within the various ranges cited in this document are envisioned as a part of the invention, without the need for each specific value to be recited.

Another object of the invention is to provide such a dietary supplement comprising a phytochemical composition comprising 5% to 70% turmeric, 3% to 50% ginger, 0.5% to 50% cinnamon, 0.05% to 30% capsicum, 0.5% to 15% grape seed extract, and 0.5% to 15% grape skin extract.

Another object of the invention is to provide such a dietary supplement comprising a phytochemical composition comprising 10% to 50% turmeric, 5% to 30% ginger, 1% to 40% cinnamon, and 0.1% to 5% capsicum.

Another object of the invention is to provide such a dietary supplement comprising a phytochemical composition comprising 10% to 50% turmeric, 5% to 30% ginger, 1% to 40% cinnamon, 0.1% to 5% capsicum, 1% to 10% grape seed extract, and 1% to 10% grape skin extract.

Embodiments of the present invention include any of the dietary supplements described above, which further comprise at least one emulsifier that increases the aqueous solubility of one or more spices in the phytochemical composition selected from turmeric, ginger, cinnamon, and capsicum.

Another object of the present invention is to provide an orally administered phytochemical composition comprising an effective inflammation-reducing amount of a mixture comprising 5% to 70% turmeric, 3% to 50% ginger, 0.5% to 50% cinnamon, 0.05% to 30% capsicum, 0.5% to 15% grape seed extract, and 0.5% to 15% grape skin extract.

Another object of the present invention is to provide an orally administered phytochemical composition comprising an effective inflammation-reducing amount of a mixture comprising 10% to 50% turmeric, 5% to 30% ginger, 1% to 40% cinnamon, and 0.1% to 5% capsicum.

Another object of the present invention is to provide an orally administered phytochemical composition comprising an effective inflammation-reducing amount of a mixture comprising 10% to 50% turmeric, 5% to 30% ginger, 1% to 40% cinnamon, 0.1% to 5% capsicum, 1% to 10% grape seed extract, and 1% to 10% grape skin extract.

Embodiments of the present invention include any of the orally administered phytochemical compositions described above, which further comprise at least one emulsifier that increases the aqueous solubility of one or more spices in the phytochemical composition selected from turmeric, ginger, cinnamon, and capsicum.

It is also an object of the invention to provide dietary supplements and phytochemical compositions formulated for oral administration, which dietary supplements and phytochemical compositions consist essentially of 5% to 70% turmeric, 3% to 50% ginger, 0.5% to 50% cinnamon, and 0.05% to 30% capsicum.

Another object of the invention is to provide dietary supplements and phytochemical compositions formulated for oral administration, which dietary supplements and phytochemical compositions consist essentially of 5% to 70% turmeric, 3% to 50% ginger, 0.5% to 50% cinnamon, 0.05% to 30% capsicum, 0.5% to 15% grape seed extract, and 0.5% to 15% grape skin extract.

Another object of the invention is to provide dietary supplements and phytochemical compositions formulated for oral administration, which dietary supplements and phytochemical compositions consist essentially of 10% to 50% turmeric, 5% to 30% ginger, 1% to 40% cinnamon, and 0.1% to 5% capsicum.

Another object of the invention is to provide dietary supplements and phytochemical compositions formulated for oral administration, which dietary supplements and phytochemical compositions consist essentially of 10% to 50% turmeric, 5% to 30% ginger, 1% to 40% cinnamon, 0.1% to 5% capsicum, 1% to 10% grape seed extract, and 1% to 10% grape skin extract.

It is also an object of the invention to provide dietary supplements and phytochemical compositions formulated for oral administration, which dietary supplements and phytochemical compositions consist essentially of a mixture of two or more phytochemical constituents selected from the group consisting of a spice, a partially purified phytochemical, and a fully purified phytochemical, which phytochemical constituents act together synergistically to change the level of a biomarker of mammalian cells in vitro in a manner that is correlated with downregulation of an inflammatory response of the cells.

It is also an object of the invention to provide dietary supplements and phytochemical compositions formulated for oral administration, which dietary supplements and phytochemical compositions consist essentially of a mixture of two or more phytochemical constituents selected from the group consisting of a spice, a partially purified phytochemical, and a fully purified phytochemical, which phytochemical constituents act together synergistically to change the level of a biomarker of a mammal in a manner that is correlated with downregulation of inflammation in the mammal following administration of the dietary supplement or phytochemical composition.

Embodiments of the present invention include any of the dietary supplements and phytochemical compositions formulated for oral administration described above, which further contain at least one emulsifier that increases the aqueous solubility of one or more spices in the dietary supplement or phytochemical composition.

The present invention provides methods for promoting wellness, good health, and/or fitness, methods for reducing or alleviating undesirable effects of inflammation, and methods for preventing undesirable effects of inflammation in a mammalian subject. The foregoing methods comprise orally administering to a mammalian subject an amount of a dietary supplement comprising a phytochemical composition comprising two or more phytochemical constituents selected from the group consisting of a spice, a partially purified phytochemical, and a fully purified phytochemical, wherein the combination of phytochemical constituents in the phytochemical composition effectively and synergistically downregulates inflammation in the subject. The foregoing methods also include methods consisting of orally administering to a mammalian subject an amount of a dietary supplement consisting essentially of a mixture of two or more phytochemical constituents selected from the group consisting of a spice, a partially purified phytochemical, and a fully purified phytochemical, which phytochemical constituents act together effectively and synergistically to downregulate inflammation in the subject.

Embodiments of these methods comprise orally administering to the subject a dietary supplement comprising a phytochemical composition comprising 5% to 70% turmeric, 3% to 50% ginger, 0.5% to 50% cinnamon, and 0.05% to 30% capsicum.

Embodiments of these methods also comprise orally administering to the subject a dietary supplement comprising a phytochemical composition comprising 5% to 70% turmeric, 3% to 50% ginger, 0.5% to 50% cinnamon, 0.05% to 30% capsicum, 0.5% to 15% grape seed extract, and 0.5% to 15% grape skin extract.

Embodiments of these methods also comprise orally administering to the subject a dietary supplement comprising a phytochemical composition comprising 10% to 50% turmeric, 5% to 30% ginger, 1% to 40% cinnamon, and 0.1% to 5% capsicum.

Embodiments of these methods also comprise orally administering to the subject a dietary supplement comprising a phytochemical composition comprising 10% to 50% turmeric, 5% to 30% ginger, 1% to 40% cinnamon, 0.1% to 5% capsicum, 1% to 10% grape seed extract, and 1% to 10% grape skin extract.

In addition, embodiments of these methods comprise orally administering to the subject a dietary supplement consisting essentially of 5% to 70% turmeric, 3% to 50% ginger, 0.5% to 50% cinnamon, and 0.05% to 30% capsicum.

Embodiments of these methods also comprise orally administering to the subject a dietary supplement consisting essentially of a phytochemical composition comprising 5% to 70% turmeric, 3% to 50% ginger, 0.5% to 50% cinnamon, 0.05% to 30% capsicum, 0.5% to 15% grape seed extract, and 0.5% to 15% grape skin extract.

Embodiments of these methods also comprise orally administering to the subject a dietary supplement consisting essentially of a phytochemical composition comprising 10% to 50% turmeric, 5% to 30% ginger, 1% to 40% cinnamon, and 0.1% to 5% capsicum.

Embodiments of these methods also comprise orally administering to the subject a dietary supplement consisting essentially of a phytochemical composition comprising 10% to 50% turmeric, 5% to 30% ginger, 1% to 40% cinnamon, 0.1% to 5% capsicum, 1% to 10% grape seed extract, and 1% to 10% grape skin extract.

Embodiments of the foregoing methods of the invention comprise orally administering to a human subject a daily dose of from 50 to 6000 mg of the dietary supplement.

It is another object of the present invention to provide a method for manufacturing a phytochemical composition containing a mixture of two or more phytochemical constituents selected from the group consisting of a spice, a partially purified phytochemical, and a fully purified phytochemical, which phytochemical constituents effectively and synergistically downregulate inflammation in a mammalian subject. Such phytochemical compositions are suitable for use as or in dietary supplements and other orally administered compositions of the present invention.

Accordingly, in one embodiment, the invention provides a method for manufacturing a phytochemical composition for reducing or alleviating, or for preventing, undesirable effects of inflammation in a subject, comprising admixing phytochemical preparations to yield an admixture suitable for oral administration that effectively downregulates inflammation and inflammatory response in a mammalian subject, wherein the admixture comprises 5% to 70% turmeric, 3% to 50% ginger, 0.5% to 50% cinnamon, and 0.05% to 30% capsicum.

Another embodiment of the invention provides a method for manufacturing a phytochemical composition comprising admixing phytochemical preparations to yield an admixture that comprises 5% to 70% turmeric, 3% to 50% ginger, 0.5% to 50% cinnamon, 0.05% to 30% capsicum, 0.5% to 15% grape seed extract, and 0.5% to 15% grape skin extract.

Another embodiment of the invention provides a method for manufacturing a phytochemical composition comprising admixing phytochemical preparations to yield an admixture that comprises 10% to 50% turmeric, 5% to 30% ginger, 1% to 40% cinnamon, and 0.1% to 5% capsicum.

Another embodiment of the invention provides a method for manufacturing a phytochemical composition comprising admixing phytochemical preparations to yield an admixture that comprises 10% to 50% turmeric, 5% to 30% ginger, 1% to 40% cinnamon, 0.1% to 5% capsicum, 1% to 10% grape seed extract, and 1% to 10% grape skin extract.

Effective embodiments of the invention include any of the methods for manufacturing a phytochemical composition described above, wherein the orally administered phytochemical composition further comprises at least one emulsifier that increases the aqueous solubility of one or more spices in the phytochemical composition selected from turmeric, ginger, cinnamon, and capsicum.

A further object of the invention is to provide a method for identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically downregulate inflammation in a mammal, the method comprising: (i) incubating mammalian cells in vitro in solution containing both at least one phytochemical of said food and the phytochemical composition comprising at least one phytochemical constituent selected from the group consisting of a spice, a partially purified phytochemical, and a fully purified phytochemical, (ii) assaying to detect a change in the level of a biomarker, which change is correlated with downregulation of an inflammatory response of the cells, and (iii) identifying a phytochemical composition comprising at least one phytochemical of said food and at least one phytochemical constituent selected from the group consisting of a spice, a partially purified phytochemical, and a fully purified phytochemical, that synergistically effect a change in the level of the biomarker in a manner that is correlated with downregulation of an inflammatory response of the cells.

In practicing the above-described in vitro method for identifying a medical food composition, the biomarker can be, but is not necessarily, selected from the group consisting of IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), and histone deacetylase (HDAC).

Another object of the invention is to provide a method for identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically downregulate inflammation in a mammal, the method comprising: (i) orally administering to a mammal a supplemented food composition comprising a food containing at least one phytochemical, and further comprising a phytochemical composition comprising at least one phytochemical constituent selected from the group consisting of a spice, a partially purified phytochemical, and a fully purified phytochemical, (ii) assaying to detect a change in the level of a bio marker, which change is correlated with downregulation of inflammation in the mammal following administration of the supplemented food composition, and (iii) identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically effect a change in the level of the biomarker in a manner that is correlated with downregulation of inflammation in the mammal.

In practicing the above-described in vivo method for identifying a medical food composition, the biomarker can be selected from the group consisting of C-reactive protein (CRP), IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), histone deacetylase (HDAC), intercellular adhesion molecule 1 (ICAM 1), myeloperoxidase (MPO), and soluble CD40 ligand (sCD40L).

It is also an object of the present invention to provide a method for preparing a medical food composition comprising a food containing at least one phytochemical and further comprising a phytochemical composition comprising at least one phytochemical constituent, which medical food composition downregulates inflammation in a mammal, the method comprising: preparing a medical food composition comprising both a food containing at least one phytochemical, and further comprising a phytochemical composition comprising at least one phytochemical constituent selected from the group consisting of a spice, a partially purified phytochemical, and a fully purified phytochemical, wherein the combination of the food and in the phytochemical composition synergistically downregulate inflammation in a mammal, and wherein said food and said phytochemical composition are present in the medical food composition in amounts effective to provide synergistic downregulation of inflammation in a mammal.

It is also an object of the present invention to provide methods for promoting wellness, good health, and fitness, methods for reducing or alleviating undesirable effects of inflammation, and methods for preventing undesirable effects of inflammation in a mammalian subject, which methods comprise orally administering to the subject a medical food composition comprising a food containing at least one phytochemical and further comprising a phytochemical composition comprising at least one phytochemical constituent selected from the group consisting of a spice, a partially purified phytochemical, and a fully purified phytochemical, wherein the combination of phytochemical constituents in the medical food composition effectively and synergistically downregulates inflammation in the subject.

### DETAILED DESCRIPTION OF THE DRAWINGS

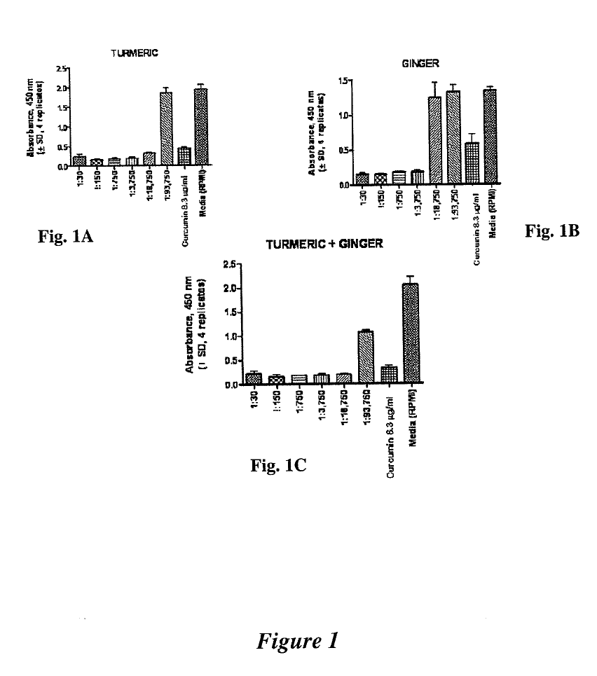

FIG. 1A—Turmeric extract at dilutions of from 1:30 to 1:18,750 inhibited LPS-stimulated expression of IL-8 by PBMCs in vitro (p<0.05).

FIG. 1B—Ginger extract at dilutions of from 1:30 to 1:3,750 inhibited LPS-stimulated expression of IL-8 by PBMCs in vitro (p<0.05).

FIG. 1C—A 1:1 combination of turmeric and ginger synergistically inhibited LPS-stimulated expression of IL-8 by PBMCs in vitro at dilutions as high as 1:93,750 (p<0.05).

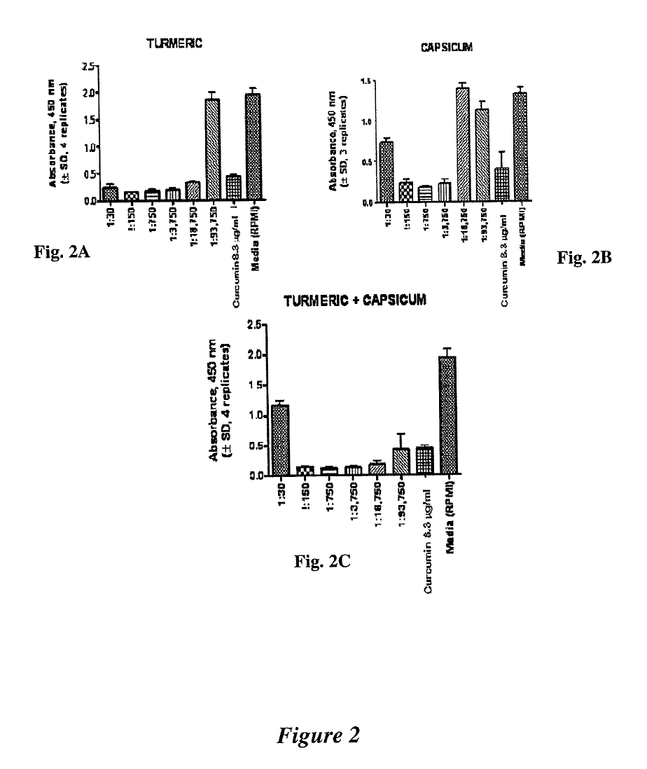

FIG. 2A—Turmeric extract at dilutions of from 1:30 to 1:18,750 inhibited LPS-stimulated expression of IL-8 by PBMCs in vitro (p<0.05).

FIG. 2B—Capsicum extract at dilutions of 1:60, 1:120 and 1:3,750 inhibited LPS-stimulated expression of IL-8 by PBMCs in vitro (p<0.05); at dilution of 1:30 only partial inhibition was observed.

FIG. 2C—A 1:1 combination of turmeric and capsicum synergistically inhibited LPS-stimulated expression of IL-8 by PBMCs in vitro at dilutions as high as 1:93,750 (p<0.05).

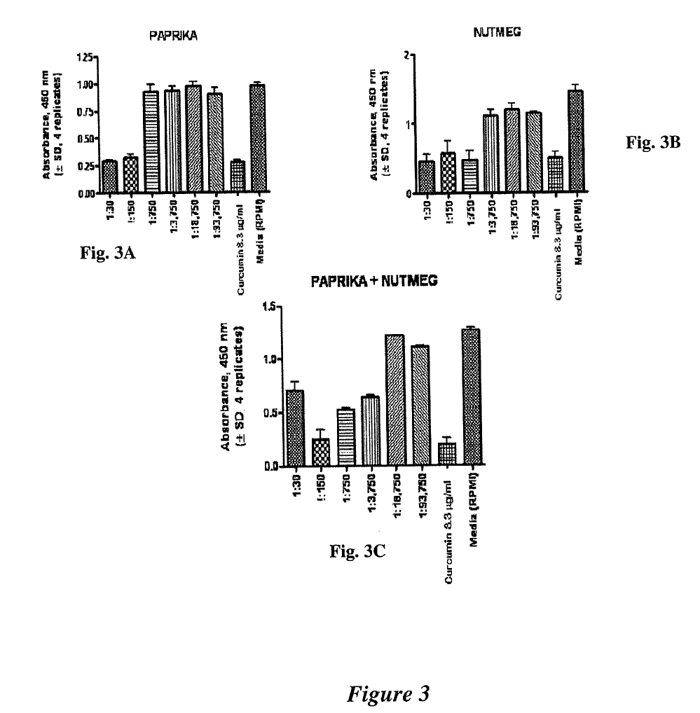

FIG. 3A—Paprika extract at dilutions of 1:30 and 1:150 inhibited LPS-stimulated expression of IL-8 by PBMCs in vitro (p<0.05).

FIG. 3B—Nutmeg extract at dilutions of 1:30, 1:150, and 1:750 significantly inhibited LPS-stimulated expression of IL-8 by PBMCs in vitro (p<0.05).

FIG. 3C—A 1:1 combination of paprika and nutmeg synergistically inhibited LPS-stimulated expression of IL-8 by PBMCs in vitro at a dilution of 1:3,750 (p<0.05).

### DETAILED DESCRIPTION OF THE INVENTION

Reference will now be made in detail to various exemplary embodiments of the invention. It is to be understood that the following discussion of exemplary embodiments is not intended as a limitation on the invention, as broadly disclosed herein. Rather, the following discussion is provided to give the reader a more detailed understanding of certain aspects and features of the invention.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a spice” includes a plurality of such spices and reference to “the mixture” includes reference to one or more mixtures and equivalents thereof known to those skilled in the art, and so forth. Furthermore, the use of terms that can be described using equivalent terms include the use of those equivalent terms. Thus, for example, the use of the term “subject” is to be understood to include the terms “animal”, “human”, “patient”, and other terms used in the art to indicate one who is subject to a medical treatment. As another example, the use of the term “cancer” is to be understood to include the terms “tumor”, “neoplasia”, “aberrant growth”, and other terms used in the art to indicate cells that are replicating, proliferating, or remaining alive in an abnormal way.

Inflammation is a defense mechanism of the immune system of mammals. It is mediated by cellular and physiological responses regulated by signaling molecules released upon irritation, injury, or infection. Benefits of inflammatory responses include the destruction of invading pathogenic microorganisms and the initiation of healing processes in injured tissues. For example, a minor injury such as a stab by a splinter causes the familiar responses of local, acute inflammation. The injury causes mast cells to release histamine, which causes nearby blood vessels to dilate and become more permeable, allowing fluid containing antimicrobial proteins to leak from the blood vessels into surrounding tissue. The increased local blood supply and permeability result in redness, heat, and swelling (edema) at the site of injury. Other locally released signaling molecules (e.g., bradykinin) stimulate nerve endings to cause pain at the site. Interleukin-8 (IL-8) and other regulatory signaling molecules released by macrophages, endothelial cells, and other cells attract neutrophils and additional macrophages, which migrate though the permeable blood vessel walls into the tissue and follow a chemotactic gradient to the site of injury and infection. Neutrophils and macrophages attack pathogens by phagocytosis. Neutrophils also release an assortment of cytotoxic, oxidizing, and anti-microbial proteins that attack invading pathogens. The processes of local, acute inflammation that eliminate invading pathogens and initiate healing are complex and involve many regulatory signaling molecules, cell types, and cellular and physiological activities in addition to the basic ones described above.

Severe tissue damage or infection may lead to an acute inflammatory state affecting the whole body, i.e., systemic, acute inflammation. One systemic inflammatory response to severe infection is the release of large numbers of neutrophils and other white blood cells from the bone marrow. Fever, resulting from an increase in the body's temperature regulatory set-point, is another systemic inflammatory response. Fever can be induced by certain bacterial toxins, and by molecules called pyrogens released by activated macrophages. The often lethal condition called septic shock, characterized by very high fever, low blood flow, and low blood pressure, is an overwhelming systemic inflammatory response caused by a bacterial or fungal infection. The infecting bacteria or fungi release toxins that cause cells in the body to release cytokines that trigger systemic inflammatory responses that can have pathological effects—they cause blood vessels to dilate, decreasing blood pressure, and can cause blood to clot in tiny blood vessels inside organs. If not alleviated, these responses can ultimately lead to organ malfunction and death.

Inflammation can be classified as either acute or chronic. If the injurious agent causing acute inflammation persists, then chronic inflammation will ensue. Chronic inflammation leads to a progressive shift in the type of cells that are present at the site of inflammation, and is characterized by the dominating presence of macrophages in the injured tissue, and simultaneous destruction and healing of the tissue from the inflammatory process. Toxins that macrophages release (including reactive oxygen species) are injurious to the host's own tissues as well as to invading agents, and chronic inflammation is almost always accompanied by tissue destruction.

Like acute inflammation, chronic inflammation can be localized to a specific site in the body, or it can be systemic. Chronic systemic inflammation is a low-grade, pervasive form of inflammation that is implicated as a major causative factor for serious chronic diseases. Causes of chronic systemic inflammation can include infections, obesity, environmental toxins, drugs and tobacco, and autoimmune diseases and disorders. Periodontal infection is believed to be a major cause of chronic systemic inflammation; organic acids and bacterial and protein toxins released from dental plaque can contribute to a low-grade chronic systemic inflammation in tissues far from the mouth. Although chronic systemic inflammation and local, acute inflammation share the same biochemical processes, chronic systemic inflammation is not restricted to a specific tissue or organ, but instead can involve the endothelial lining of blood vessels and other tissue types throughout the body. For example, systemic inflammation in response to tobacco toxins is known to cause damage to the arterial endothelium, thereby aggravating the development of atherosclerosis. Autoimmune diseases and disorders are pathological conditions in which the immune system reacts inappropriately to normal tissues; they include rheumatoid arthritis and lupus, vary widely in their intensity, and usually precipitate serious inflammatory-mediated tissue damage. The injurious effects of chronic systemic inflammation are far reaching, causing damage to the cardiovascular, nervous, endocrine, and other systems. This damage may, in turn, precipitate or exacerbate a wide variety of other pathological processes. For example, atherosclerosis involves a chronic inflammatory response in the walls of arteries that results in formation of plaques containing fats, cholesterol, and macrophages in the arteries that can severely obstruct blood flow. Chronic systemic inflammation also destabilizes the cholesterol deposits in arteries, leading to thrombosis, heart attacks, and strokes. Chronic systemic inflammation is implicated in the development of, and/or the injuries resulting from, many common diseases and disorders, including but not limited to cardiovascular disease, insulin resistance and diabetes, hay fever and other allergies, asthma, Alzheimer's disease, anemia, rheumatoid arthritis and other autoimmune diseases, cancers, some forms of depression, and even sleep disorders.

Many foods, herbs, spices, and medicinal plants are known to contain phytochemicals that promote good health and longevity, and/or have therapeutic properties. For example, medicinal use of turmeric as an anti-inflammatory agent has a 5000 year history in Ayurvedic medical tradition of the Indian sub-continent. Many of these phytochemicals are produced by plants as a hormetic response to stress (see Gerber et al., 1999). Research within the last century has shown that curcumin, the active component of turmeric, can suppress psoriasis, alleviate Crohn's disease, and improve arthritis-associated symptoms. Scientists have also identified a growing number of other phytochemicals in foods, herbs and spices with beneficial properties (Aggarwal and Shishodia 2004). Research efforts to date have largely focused on identifying and characterizing individual phytochemicals with health-promoting and therapeutic properties. A limitation of the use of many individual phytochemicals is that their relatively weak physiological or pharmacological activities generally lack the potency to achieve immune regulatory efficacy, and the doses required are much higher than can be achieved as part of normal diet. When more potent phytochemicals are purified and used individually as drugs, they are subject to the same single-site-of-action potency and toxicity limitations associated with pharmaceutical drugs.

While much effort has been expended identifying and characterizing individual phytochemicals with health-promoting and therapeutic properties, there has been little research to identify combinations of foods, herbs, and spices that act synergistically to promote good health, well-being, and longevity, and/or provide therapeutic benefits. The biochemical regulatory and metabolic pathways that mediate cellular immune and inflammatory responses are complex and have a high degree of redundancy and inter-relatedness. Interventions or treatments that use individual drugs or phytochemicals to modulate the immune system and/or to downregulate inflammation are not as likely to be as effective as treatments that target multiple elements of the biochemical regulatory and metabolic pathways that mediate cellular immune and inflammatory responses. The ability of phytochemical blends to target a diversity of sites of action, albeit with less potency than pharmaceutical drugs, allows desirable regulation of pathological immunity by effecting multiple biochemical pathways to defeat the redundancies that are inherit in mechanisms of immune response. For example 20% inhibition of a half dozen sites of action involving multiple biochemical pathways by a phytochemical blend can be significantly more efficacious and less toxic than 80% inhibition of a single site of action by a pharmaceutical drug (see Underwood, 2005).

The present invention provides methods that identify phytochemical blends—combinations of phytochemical-containing foods, herbs and spices—that act synergistically to modulate immune responses, downregulate inflammation, and promote wellness, good health, and/or fitness in a mammal. Rather than assay for interactions of phytochemicals with specific molecular targets in cells of the immune system, the methods of the invention rely on assays that detect changes in the levels of biomarkers that are correlated with downregulation of an inflammatory response. Working examples are described that demonstrate using the method of the invention to identify three different combinations of spices that synergistically alter the level of a biomarker in a manner that is correlated with downregulation of an inflammatory response of cells of the mammalian immune system. The present invention further provides a dietary supplement and an orally administered, synergistically acting phytochemical composition for reducing or preventing inflammation and promoting wellness, good health, and/or fitness of a mammalian subject, and a method for reducing or preventing inflammation in a mammal by orally administering such a synergistically acting phytochemical composition.

### Definitions

Plant material: As used herein, “plant material” broadly refers to any part of a plant, e.g., root, stem, leaf, flower, or seed, as well as to a collection or mixture of multiple plant parts. Plant material of the invention containing multiple plant parts can include complete or partial plant parts of the same or of different types; e.g., whole leaves or flower petals, or a mixture of leaves and flower petals. A plant part in plant material of the invention can be intact or non-intact, e.g., crushed, broken up, chopped up, ground up, sliced, powdered, shredded, grated, etc., and can be fresh, dried, cooked or uncooked, or otherwise physically processed or prepared. The plant parts in plant material of the invention can be of the same or of different plant species.

Phytochemical: As used herein, “phytochemical” broadly refers to a bioactive, non-nutrient chemical compound that occurs naturally in a plant. Many phytochemicals have been shown or are believed to provide health benefits. A large number of phytochemicals, such as lycopene in tomatoes and the catechin polyphenols in green tea, have antioxidant properties and can protect against “free radicals” that can damage healthy cells. Taxol (paclitaxel), an important cancer drug, is a phytochemical initially extracted and purified from the Pacific yew tree. There is evidence from laboratory studies that other phytochemicals in fruits and vegetables can reduce the risk of cancer. 3,3′-Diindolylmethane, an anticarcinogen compound derived from the digestion of indole-3-carbinol in Brassica vegetables (e.g., broccoli, kale, Brussels sprouts), is currently in several clinical trials for prostate cancer (“Diindolylmethane Clinical Trials.” ClinicalTrials.gov, US National Institutes of Health, Department of Health and Human Services. 2010.)

Phytochemical composition: As used herein, “phytochemical composition” refers to a composition comprising least one phytochemical. A phytochemical composition of the invention may be a composition consisting entirely of a single phytochemical or of multiple phytochemicals; i.e., it may be a sample of a purified phytochemical, or a mixture of two or more purified phytochemicals. A phytochemical composition of the invention may also be a composition that contains only a very small amount of a phytochemical. For example, one or more of the phytochemicals in a phytochemical composition may be present in an amount that is from 0.000001% to 0.00001%, or from 0.00001% to 0.0001%, or from 0.0001% to 0.001%, or from 0.001% to 0.01%, or from 0.01% to 0.1%, or from 0.1% to 1% or more, by weight, of the matter that makes up the phytochemical composition. A phytochemical composition of the invention can contain two or more phytochemicals that are present in very different concentrations. For example, one phytochemical may constitute 20% by weight, and another phytochemical may constitute only 0.02% by weight, of the phytochemical composition. In addition to containing at least one phytochemical, a phytochemical composition of the invention can contain plant material; e.g., material of the plant that that produced the phytochemical, or material of a different plant of the same or different species. A phytochemical composition of the invention can contain a food or a beverage. A phytochemical composition of the invention can contain one or more components selected from inert diluents, edible carriers, pharmaceutically acceptable vehicles, pharmaceutically acceptable excipients, pharmaceutically acceptable adjuvants, and pharmaceutically acceptable carriers, as described below with regard to phytochemical composition formulations suitable for oral administration.

Phytochemical blend: As used herein, “phytochemical blend” refers to a phytochemical composition comprising two or more different phytochemicals.

Phytochemical constituent: As used herein, “phytochemical constituent” refers to a phytochemical-containing component of a phytochemical composition. Non-limiting examples of a phytochemical constituent of the invention include, but are not limited to, a partially purified phytochemical, a fully purified phytochemical, a whole spice, and a spice extract.

Spice: As used herein, “spice” broadly refers to any plant material that is used as a flavoring agent in foods or beverages. Spices suitable for use in the present invention include plant materials consisting of or obtained from seeds, roots, bark, flowers, flower buds, fruits, stems, and leaves. Ginger, for example, is a spice obtained from underground stems called rhizomes. Spices suitable for use in the present invention include plant materials consisting of or obtained from leaves and tender stems that are commonly referred to as herbs; e.g., leaves of oregano, thyme, sage, or mint plants. The term “spice” as used herein also includes plant material used for flavoring foods that is also used as a food itself. Examples of such spices include garlic, onion, and chili pepper, which are added in small amounts to flavor food preparations, and are also eaten in bulk as foods. Spices suitable for use in the present invention can be used in either a fresh or dried state. Spices suitable for use in the present invention can be used as whole spices or as spice extracts. Insofar as a spice contains two or more different phytochemicals, it also is itself a phytochemical blend.

Whole spice: As used herein, “whole spice” refers to the complete or nearly complete plant material constituting a spice, no matter how it is prepared; e.g., regardless of whether it is intact or crushed, macerated, or powdered, or whether it is fresh or dried. For example, rhizomes of the turmeric plant (Curcuma longa) are boiled, dried and ground to produce orange-yellow powder, also called turmeric, which is an ingredient of curry powder. Turmeric rhizome is also chopped, grated, or crushed and used fresh, much like ginger, to impart flavor to foods. In some areas, leaves of the turmeric plant are chopped or ground and used as flavoring agents. Foods are also wrapped in whole leaves of the turmeric plant before they are cooked, a practice that imparts a distinct flavor to the food. While the physical form of the turmeric plant material in each of these examples is different, the complete plant material (from rhizome or leaf) is used in each case, and these are all examples of using turmeric plant material as a “whole spice” as the term is used herein. In analogous fashion, various parts of other plants, in various physical forms, can also be used as a “whole spice” as the term is used herein.

Spice extract: As used herein, “spice extract” refers to a composition containing phytochemicals having odor, flavor, and/or color qualities of a spice that are separated or extracted from the complete plant material constituting a spice, usually by a chemical process. Spice extracts have important commercial use, and many methods for preparing spice extracts have been described. For example, U.S. Pat. No. 4,158,708, incorporated by reference herein in its entirety, describes preparing a spice extract comprising an aromatic fraction A consisting of the gases given off during grinding, an aromatic fraction B extracted by treating the spice with an apolar organic solvent, and an aromatic fraction C extracted by treating the spice with at least one polar solvent. U.S. Pat. No. 5,120,558, incorporated by reference herein in its entirety, reviews various known methods for preparing spice extracts, and describes a process for extracting spices and herbs using supercritical fluid carbon dioxide. Spice extracts called oleoresins are produced by the solvent extraction of a ground spice, with subsequent solvent removal and occasionally purifying steps to remove unwanted components, such as waxes and chlorophyll. U.S. Pat. No. 4,681,769, incorporated by reference herein in its entirety, describes methods for preparing extracts of natural flavor and color elements from ground spice using edible oils as a solvent. A spice extract called essential oil is a volatile oil obtained from a spice, most commonly by steam distillation, which usually has the characteristic (essential) odor and flavor of the spice. Liquid spice extracts are produced by dissolving a spice oleoresins or essential oils in a vegetable oil or alcohol. Aqueous liquid spice preparations are manufactured by emulsifying spice oleoresins or essential oils in water with emulsifier(s) and stabilizer(s). Dissolvable, granularized spice preparations are produced by absorption of spice oleoresins or essential oils onto dissolvable particles of edible material such as glucose, dextrin, or salt. Methods for making liquid spice extracts and granularized spice extract preparations are well known and are in wide commercial use. Insofar as a spice extract contains two or more different phytochemicals, it too is a phytochemical blend.

Paprika: As used herein, “paprika” refers to a spice made from ground or powdered dried fruits of Capsicum annuum. Cultivars of Capsicum annuum include “sweet” bell peppers as well as “hot” chili peppers. Paprika can be made with any of the Capsicum annuum cultivars, or with a mixture of different cultivars; hence the flavor of paprika can range from sweet (mild, not hot) to spicy (hot).

Capsicum: As used herein, “capsicum” refers to a spice made from edible fruit of a plant of the genus Capsicum, a genus of flowering plants in the nightshade family, Solanaceae. Species of the Capsicum genus that produce edible fruit from which a spice of the present invention can be made include, but are not limited to, Capsicum annuum, Capsicum chinense, and Capsicum frutescens. For example, synergistic downregulation of inflammation is provided by a phytochemical blend of the present invention that contains turmeric and cayenne pepper, a cultivar of Capsicum annuum.

Synergy and synergistic: As used herein, “synergy” and “synergistic” refer to a biological activity of two or more phytochemical compositions present in a mixture or acting together in a single assay sample that is greater than the sum of the biological activities of the individual phytochemical compositions acting separately under the same conditions.

Mammal: As used herein, the term “mammal” includes humans and all other animals of the taxonomic class Mammalia, e.g., domesticated mammals such as cattle, sheep, goats, pigs, horses, and llamas, pet mammals such as dogs, cats, ferrets, and rabbits, and laboratory research mammals, such as rats, mice, non-human primates, and guinea pigs.

Biomarker: As used herein, “biomarker” refers to a biological molecule, biochemical feature, or cellular process or activity, e.g., mRNA synthesis or cell proliferation, that is a sign of a normal or abnormal process, or of a condition or disease, and can be used to measure the effects of treatment or the progress of a disease or condition.

Downregulation: As used herein, “downregulation” of a biological activity refers to reducing, inhibiting, suppressing, or lowering the level, degree, or amount of the biological activity. The process of downregulation, as used herein, can be mediated by any single or combination of molecular, metabolic, and/or physiological mechanisms. For example, downregulation of a biological activity can be mediated by altering the level of expression of one or more genes, e.g., by altering the rate of transcription, by altering the rate or pattern of processing of hnRNA to mRNA, or by altering mRNA half-life. Downregulation of a biological activity can also be mediated by effecting cleavage or degradation of a protein, or by otherwise altering its half-life, or by chemically altering the catalytic or functional activity of one or more proteins, e.g., by phosphorylation or dephosphorylation, acetylation or deacetylation, glycosylation, etc. Depending on the biological activity being affected and the metabolic milieu, downregulation might be mediated either by increasing or decreasing the expression of one or more genes or the activities of one or more proteins, or by a complex combination of these mechanisms. For example, downregulation of a biological activity might be effected by activating a transcription factor that induces expression of a gene that encodes an inhibitor that inhibits and thereby downregulates a biological activity of interest.

Ranges: It is to be understood that numerical ranges disclosed herein include each value at the upper and lower end of the disclosed range. Each range also includes each numerical value within the range. Those of skill in the art will immediately understand that each particular value within the range is inherently disclosed by disclosure of the range's upper and lower values, and that a specific disclosure of each value within each range is not necessary for understanding that each value is inherently disclosed. Thus, for example, a range of 15% -70% inherently provides written description of a range of 16% -70%, 20% -65%, 30% -50%, etc. Specific disclosure of all of the permutations of ranges presented herein would be excessively burdensome on the inventor and is not necessary in view of the knowledge and understanding of the skilled artisan.

The present invention provides methods for identifying a phytochemical composition comprising two or more phytochemical constituents that synergistically downregulate inflammation in a mammal, and for making dietary supplements containing such phytochemical compositions that can be administered to promote wellness, good health, and/or fitness in a mammal.

The present invention provides a method for identifying a phytochemical composition comprising two or more phytochemical constituents that synergistically downregulate inflammation in a mammal, the method comprising: (i) contacting mammalian cells with a phytochemical composition comprising two or more phytochemical constituents selected from the group consisting of a spice, a partially purified phytochemical, and a fully purified phytochemical, (ii) assaying to detect a change in the level of a biomarker, which change is correlated with downregulation of an inflammatory response of the cells, (iii) identifying a phytochemical composition comprising two or more phytochemical constituents that synergistically effect a change in the level of the biomarker in a manner that is correlated with downregulation of an inflammatory response of the cells.

The above-described method of the invention can be performed effectively in vitro, using a method in which step (i) comprises incubating mammalian cells in vitro in solution containing a phytochemical composition comprising two or more phytochemical constituents selected from the group consisting of a spice, a partially purified phytochemical, and a fully purified phytochemical, and step (ii) comprises assaying to detect a change in the level of a biomarker, which change is correlated with downregulation of an inflammatory response of the cells. Biomarkers suitable for use in performing the method in vitro include but are not limited to IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), and histone deacetylase (HDAC). As used herein, the terms IL-1, IL-6, IL-8, and IL-10 refer to all pro-inflammatory and inflammation-correlated forms of these interleukins, including, IL-1 alpha and IL-1 beta.

The above-described method of the invention can also be performed effectively in vivo, using a method in which step (i) comprises orally administering to a mammal a phytochemical composition comprising two or more phytochemical constituents selected from the group consisting of a spice, a partially purified phytochemical, and a fully purified phytochemical, and step (ii) comprises assaying to detect a change in the level of a biomarker, which change is correlated with downregulation of inflammation in the mammal following administration of the phytochemical composition. The method can also be performed effectively using a combination of in vivo and ex vivo methodologies. For example, the method can be performed using step (i) comprising orally administering to a mammal a phytochemical composition comprising two or more phytochemical constituents selected from the group consisting of a spice, a partially purified phytochemical, and a fully purified phytochemical, and then isolating cells from the mammal and carrying out step (ii) ex vivo comprising assaying to detect a change in the level of a biomarker, which change is correlated with downregulation of an inflammatory response of the cells that is in turn correlated with downregulation of inflammation in the mammal following administration of the phytochemical composition. Biomarkers suitable for use in performing the method in vivo or by a combination of in vivo and ex vivo methodologies include but are not limited to C-reactive protein (CRP), interleukin IL-1, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), histone deacetylase (HDAC), intercellular adhesion molecule 1 (ICAM 1), myeloperoxidase (MPO), and soluble CD40 ligand (sCD40L).

These and all other methods of the invention can be practiced using any mammalian cells that express or produce a biomarker at a level that is correlated with the level of an inflammatory response of the cells under the conditions used. In one embodiment of the invention, the cells are human peripheral blood mononuclear cells (PBMCs).

In the above-described embodiments, as well as in all other embodiments of the invention, the spices of the invention can be either whole spices or spice extracts. As defined herein, a spice of the invention can be in the form of a liquid spice extract comprising at least one emulsifier that increases the aqueous solubility of the extract.

Phytochemical compositions that are suitable for use in all embodiments of the invention include but are not limited to phytochemical compositions comprising a synergistically acting combination of spices selected from (a) turmeric and ginger, (b) turmeric and capsicum, and (c) paprika and nutmeg.

Incubating mammalian cells with a phytochemical composition comprising curcumin and resveratrol has also been found to synergistically reduce IL-8 production by the cells following stimulation with sulfur mustard. Accordingly, phytochemical compositions that are suitable for use in all embodiments of the invention include but are not limited to phytochemical compositions comprising a synergistically acting combination of turmeric and resveratrol.

Once a phytochemical composition comprising two or more phytochemical constituents (purified phytochemicals, spices, or a combination thereof) that synergistically downregulate mammalian inflammation is identified, it is desirable to determine those ratios of the synergistically acting constituents that provide a relatively high degree of synergistic downregulation of inflammation in a mammal. The present invention provides methods for identifying an optimized phytochemical composition comprising two or more phytochemical constituents that are present in a ratio selected to provide a desired degree of synergistic downregulation of inflammation and inflammatory response in a mammal.

One embodiment of such a method is an in vitro method comprising the steps of: (i) preparing multiple phytochemical compositions comprising various selected ratios of two or more phytochemical constituents selected from the group consisting of a spice, a partially purified phytochemical, and a fully purified phytochemical, (ii) incubating mammalian cells in vitro in solutions, each of which contains one of the multiple phytochemical compositions of step (i), (iii) assaying each cell sample to detect a change in the level of a biomarker, which change is correlated with downregulation of an inflammatory response of the cells, and (iv) identifying a phytochemical composition comprising two or more phytochemical constituents that synergistically downregulate an inflammatory response of mammalian cells, which constituents are present in a ratio that provides a desired level of relative synergistic activity.

Biomarkers that are suitable for practicing the method of the invention in vitro are described above. In addition, other known biomarkers, the levels of which are known to correlate with the level of an inflammatory response of mammalian cells, can also be used effectively.

Another embodiment of a method of the invention for identifying an optimized phytochemical composition is an in vivo method comprising the steps of: (i) preparing multiple phytochemical compositions comprising various selected ratios of two or more phytochemical constituents selected from the group consisting of a spice, a partially purified phytochemical, and a fully purified phytochemical, (ii) orally administering each of the multiple phytochemical compositions to a mammalian subject, and assaying to detect a change in the level of a biomarker, which change is correlated with downregulation of inflammation in the mammal following administration of the phytochemical composition, and (iii) identifying a phytochemical composition comprising two or more phytochemical constituents that synergistically downregulate inflammation in a mammal, which constituents are present in a ratio that provides a desired level of relative synergistic activity.

Biomarkers that are suitable for practicing the method of the invention in vivo are described above. Other known biomarkers, the levels of which are known to correlate with the level of inflammation in a mammal, can also be used effectively.

The present invention provides methods for making a dietary supplement that contains a phytochemical composition that downregulates inflammation in a mammal; one embodiment of such a method comprises: preparing a dietary supplement comprising a phytochemical composition comprising two or more phytochemical constituents selected from the group consisting of a spice, a partially purified phytochemical, and a fully purified phytochemical, which combination of phytochemical constituents synergistically changes the level of a biomarker of mammalian cells in vitro in a manner that is correlated with downregulation of an inflammatory response of the cells, wherein said two or more phytochemical constituents are present in amounts effective to synergistically downregulate inflammation in a mammal.

Another embodiment of a method for making a dietary supplement containing a phytochemical composition that downregulates inflammation in a mammal comprises: preparing a dietary supplement comprising a phytochemical composition comprising two or more phytochemical constituents selected from the group consisting of a spice, a partially purified phytochemical, and a fully purified phytochemical, which combination of phytochemical constituents synergistically effects a change in the level of a mammalian biomarker in a manner that is correlated with downregulation of inflammation in a mammal following oral administration of the phytochemical composition, wherein said two or more phytochemical constituents are present in the dietary supplement in amounts effective to downregulate inflammation in a mammal.

The invention also provides a method for making a dietary supplement containing a phytochemical composition that downregulates inflammation in a mammal, the method comprising: preparing a dietary supplement comprising two spices selected from the group consisting of (a) turmeric and ginger, (b) turmeric and capsicum, and (c) paprika and nutmeg, in amounts effective to downregulate inflammation in a mammal following oral administration of the phytochemical composition.

The invention also provides a method for making a dietary supplement that downregulates inflammation in a mammal, the method comprising: preparing a dietary supplement consisting essentially of one or more of the pairs of synergistically acting spices selected from the group consisting of (a) turmeric and ginger, (b) turmeric and capsicum, and (c) paprika and nutmeg, in amounts effective to downregulate inflammation in a mammal following oral administration of the phytochemical composition.

The present invention provides an in vitro method for identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically downregulate inflammation in a mammal. The method comprises: (i) incubating mammalian cells in vitro in solution containing both at least one phytochemical of said food and the phytochemical composition comprising at least one phytochemical constituent selected from the group consisting of a spice, a partially purified phytochemical, and a fully purified phytochemical, (ii) assaying to detect a change in the level of a biomarker, which change is correlated with downregulation of an inflammatory response of the cells, and (iii) identifying a phytochemical composition comprising at least one phytochemical of said food and at least one phytochemical constituent selected from the group consisting of a spice, a partially purified phytochemical, and a fully purified phytochemical, that synergistically effect a change in the level of the biomarker in a manner that is correlated with downregulation of an inflammatory response of the cells.

Biomarkers that are suitable for practicing the method of the invention in vitro are described above. In addition, other known biomarkers, the levels of which are known to correlate with the level of an inflammatory response of mammalian cells, can also be used effectively.

The present invention also provides an in vivo method for identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically downregulate inflammation in a mammal. The method comprises: (i) orally administering to a mammal a supplemented food composition comprising a food containing at least one phytochemical, and further comprising a phytochemical composition comprising at least one phytochemical constituent selected from the group consisting of a spice, a partially purified phytochemical, and a fully purified phytochemical, (ii) assaying to detect a change in the level of a biomarker, which change is correlated with downregulation of inflammation in the mammal following administration of the supplemented food composition, and (iii) identifying a medical food composition comprising a food containing at least one phytochemical, and further comprising a phytochemical composition comprising at least one phytochemical constituent selected from the group consisting of a spice, a partially purified phytochemical, and a fully purified phytochemical, wherein the combination of the food and the phytochemical composition synergistically effect a change in the level of the biomarker in a manner that is correlated with downregulation of inflammation in the mammal.

Biomarkers that are suitable for practicing the method of the invention in vivo are described above. Other known biomarkers, the levels of which are known to correlate with the level of inflammation in a mammal, can also be used effectively.

A medical food composition comprising a food containing at least one phytochemical and further comprising a phytochemical composition comprising at least one phytochemical constituent, which medical food composition downregulates inflammation in a mammal, can be prepared using well-known food preparation methods. Such a medical food composition can be orally administered to a subject to promote wellness, to promote good health, or to promote fitness of the subject. Such a medical food composition can be orally administered to a subject to reduce or alleviate undesirable effects of inflammation. Such a medical food composition can also be orally administered to a subject to prevent undesirable effects of inflammation.

The present invention provides methods for promoting wellness, methods for promoting good health, and methods for promoting fitness in a mammalian subject. The present invention also provides methods for preventing inflammation in a mammalian subject, and methods for alleviating or reducing undesirable effects of inflammation in a mammalian subject. All of these methods comprise orally administering to the subject an amount of a dietary supplement comprising a phytochemical composition that effectively downregulates inflammation in the subject, wherein the phytochemical composition comprises 5% to 70% turmeric, 3% to 50% ginger, 0.5% to 50% cinnamon, and 0.05% to 30% capsicum. An effective variant of these methods comprises orally administering a dietary supplement comprising a phytochemical composition comprising 5% to 70% turmeric, 3% to 50% ginger, 0.5% to 50% cinnamon, 0.05% to 30% capsicum, and further comprising 0.5% to 15% grape seed extract, and 0.5% to 15% grape skin extract.

The aforementioned methods can also be practiced effectively by orally administering a an amount of a dietary supplement comprising a phytochemical composition that effectively downregulates inflammation in the subject, wherein the phytochemical composition comprises 10% to 50% turmeric, 5% to 30% ginger, 1% to 40% cinnamon, and 0.1% to 5% capsicum. An effective variant of this embodiment of the invention comprises orally administering a dietary supplement comprising a phytochemical composition comprising 10% to 50% turmeric, 5% to 30% ginger, 1% to 40% cinnamon, and 0.1% to 5% capsicum, and further comprising 1% to 10% grape seed extract, and 1% to 10% grape skin extract.

As described above, the present invention provides methods for promoting wellness, methods for promoting good health, and methods for promoting fitness in a mammalian subject. The present invention also provides methods for preventing inflammation in a mammalian subject, and methods for alleviating or reducing undesirable effects of inflammation in a mammalian subject. All of these methods can be practiced effectively by orally administering to the subject a dietary supplement consisting essentially of one or more of the pairs of synergistically acting spices selected from the group consisting of (a) turmeric and ginger, (b) turmeric and capsicum, and (c) paprika and nutmeg, in amounts effective to downregulate inflammation in the subject following oral administration of the phytochemical composition.

All of the aforementioned methods can also be practiced effectively by orally administering to the subject an amount of a dietary supplement containing a phytochemical composition that effectively downregulates inflammation in the subject, wherein the phytochemical composition consists essentially of 5% to 70% turmeric, 3% to 50% ginger, 0.5% to 50% cinnamon, and 0.05% to 30% capsicum. An effective variant of these methods comprises orally administering a dietary supplement containing a phytochemical composition consisting essentially of 5% to 70% turmeric, 3% to 50% ginger, 0.5% to 50% cinnamon, 0.05% to 30% capsicum, and further comprising 0.5% to 15% grape seed extract, and 0.5% to 15% grape skin extract.

The aforementioned methods can also be practiced effectively by orally administering a an amount of a dietary supplement containing a phytochemical composition that effectively downregulates inflammation in the subject, wherein the phytochemical composition consists essentially of 10% to 50% turmeric, 5% to 30% ginger, 1% to 40% cinnamon, and 0.1% to 5% capsicum. An effective variant of this embodiment of the invention comprises orally administering a dietary supplement containing a phytochemical composition consisting essentially of 10% to 50% turmeric, 5% to 30% ginger, 1% to 40% cinnamon, and 0.1% to 5% capsicum, and further comprising 1% to 10% grape seed extract, and 1% to 10% grape skin extract.

All embodiments of the invention that are methods that comprise orally administering a dietary supplement or phytochemical composition that downregulates inflammatory response or inflammation in a mammal include methods of self-administering the dietary supplement, and also include methods wherein the dietary supplement is administered to the mammalian subject by an agent other than the mammalian subject.

The methods of the invention for preventing undesirable effects of inflammation in a mammalian subject comprise orally administering an effective amount of the dietary supplement to a subject before the subject is exposed to an inflammation-eliciting agent, in order to partially or completely prevent inflammation from being elicited in the subject by the inflammation-eliciting agent.

The methods of the invention for preventing undesirable effects of inflammation in a subject, and the methods for alleviating or reducing undesirable effects of inflammation in a subject, have vital application as methods for protecting soldiers and citizens against exposure to multiple classes of toxic chemical agents, the need for which is set forth in “Homeland Security Presidential Directive/HSPD-18” Medical Countermeasures against Weapons of Mass Destruction (Bush 2007).

The above-described methods for preventing or reducing undesirable effects of inflammation in a mammalian subject can be practiced effectively in an embodiment of the invention comprising orally administering to a human subject a daily dose of the dietary supplement containing from 50 to 6000 mg of the phytochemical composition.

Embodiments of the present invention provide dietary supplements comprising a phytochemical composition comprising turmeric, ginger, cinnamon, and capsicum, or combinations of two or more of these, that effectively downregulate inflammation in a mammal. In embodiments, at least one of the spices is a spice extract. In some embodiments, the composition further includes at least one emulsifier that increases the aqueous solubility of at least one extract in the phytochemical composition.

For example, embodiments of the invention include phytochemical compositions comprising two spices selected from the group consisting of (a) turmeric and ginger, (b) turmeric and capsicum, and (c) paprika and nutmeg, in amounts effective to downregulate inflammation in a mammal following oral administration of the phytochemical composition. Exemplary embodiments comprise 5% to 70% turmeric and 3% to 50% ginger. Other exemplary embodiments comprise 5% to 70% turmeric and 0.05% to 30% capsicum. Yet other exemplary embodiments of the dietary supplement of the invention comprise a phytochemical composition comprising 5% to 70% turmeric, 3% to 50% ginger, 0.5% to 50% cinnamon, and 0.05% to 30% capsicum.

Another embodiment of the dietary supplement of the invention comprises a phytochemical composition comprising 5% to 70% turmeric, 3% to 50% ginger, 0.5% to 50% cinnamon, 0.05% to 30% capsicum, 0.5% to 15% grape seed extract, and 0.5% to 15% grape skin extract.

Another embodiment of the dietary supplement of the invention comprises a phytochemical composition comprising 10% to 50% turmeric, 5% to 30% ginger, 1% to 40% cinnamon, and 0.1% to 5% capsicum.

Another embodiment of the dietary supplement of the invention comprises a phytochemical composition comprising 10% to 50% turmeric, 5% to 30% ginger, 1% to 40% cinnamon, 0.1% to 5% capsicum, 1% to 10% grape seed extract, and 1% to 10% grape skin extract. Yet another embodiment of the dietary supplement of the invention comprises a phytochemical composition comprising 10% to 50% turmeric, 5% to 30% ginger, 1% to 40% cinnamon, 0.1% to 5% capsicum, 0.5% to 15% grape seed extract, and 0.5% to 15% grape skin extract.

Effective embodiments of the dietary supplement of the invention also include any of the dietary supplements described above, which further contain at least one emulsifier that increases the aqueous solubility of one or more spices in the phytochemical composition selected from turmeric, ginger, cinnamon, and capsicum.

In all embodiments of the dietary supplement and orally administered composition of the invention, the recited substances can be provided in the form of a fully or partially purified phytochemical, a phytochemical composition, a phytochemical blend, a spice, a whole spice, a spice extract, or any combination of these substances.

Embodiments of the present invention also provide orally administered phytochemical compositions comprising turmeric, ginger, cinnamon, and capsicum, that effectively downregulate inflammation in a mammal.

In one embodiment, the orally administered phytochemical composition of the invention comprises a phytochemical composition comprising 5% to 70% turmeric, 3% to 50% ginger, 0.5% to 50% cinnamon, and 0.05% to 30% capsicum.

In another embodiment, the orally administered phytochemical composition of the invention comprises a phytochemical composition comprising 5% to 70% turmeric, 3% to 50% ginger, 0.5% to 50% cinnamon, 0.05% to 30% capsicum, 0.5% to 15% grape seed extract, and 0.5% to 15% grape skin extract.

In another embodiment, the orally administered phytochemical composition of the invention comprises a phytochemical composition comprising 10% to 50% turmeric, 5% to 30% ginger, 1% to 40% cinnamon, and 0.1% to 5% capsicum.

In another embodiment, the orally administered phytochemical composition of the invention comprises a phytochemical composition comprising 10% to 50% turmeric, 5% to 30% ginger, 1% to 40% cinnamon, 0.1% to 5% capsicum, 1% to 10% grape seed extract, and 1% to 10% grape skin extract.

Effective embodiments of the orally administered phytochemical compositions of the invention also include any of the orally administered phytochemical compositions described above, which further contain at least one emulsifier that increases the aqueous solubility of one or more spices in the phytochemical composition selected from turmeric, ginger, cinnamon, and capsicum.

Dietary supplements and orally administered phytochemical compositions of the invention such as those described above effectively alleviate or reduce undesirable effects of inflammation in a mammal. Such dietary supplements and orally administered phytochemical compositions also prevent undesirable effects of inflammation in a mammal. In addition, such dietary supplements and orally administered phytochemical compositions promote wellness in a mammal. Such dietary supplements and orally administered phytochemical compositions also promote good health in a mammal. Such dietary supplements and orally administered phytochemical compositions also promote fitness in a mammal.

The present invention also provides processes for manufacturing dietary supplements and orally administered phytochemical compositions comprising the spices turmeric, ginger, cinnamon, and capsicum, that effectively downregulate inflammation in a mammal. Embodiments of these processes comprise admixing phytochemical preparations to yield an admixture that has the composition of one of the dietary supplements or orally administered phytochemical compositions of the invention set forth above.

### Formulations

Embodiments of the invention comprise orally administered phytochemical compositions for reducing inflammation in a mammalian subject, and oral dietary supplements comprising said orally administered phytochemical compositions, as described. Included in the present invention are embodiments wherein the orally administered phytochemical composition or the oral dietary supplement is a powder suitable for mixing and ingesting with a food. Also included in the present invention are embodiments wherein the orally administered phytochemical composition or the oral dietary supplement is a powder suitable for mixing with a suitable liquid, such as water or a hot beverage to form a tonic. The present invention also includes embodiments wherein the orally administered phytochemical composition or the oral dietary supplement is processed into an aqueous suspension, or a liquid solution.

In one embodiment, the orally administered phytochemical composition or the oral dietary supplement is processed into a dosage unit for oral administration as a tablet, capsule, gel cap, pellet (globule), or in other carrier suitable for oral administration. Such oral compositions enclosed in gelatin capsules, caplets or compressed into tablets can include an inert diluent or an edible carrier. In one embodiment, the orally administered phytochemical composition or the oral dietary supplement of the invention is delivered in an enterically coated tablet, caplet, or capsule, to further enhance stability and provide release in the intestinal tract to improve absorption.

An effective and proper amount of the can be formulated as to provide a composition that can be administered as a suitable daily oral dietary supplement. The orally administered phytochemical composition or the oral dietary supplement of the invention can be used alone or further formulated with pharmaceutically acceptable compounds, vehicles, excipients or adjuvants with a favorable delivery profile, i.e., suitable for delivery to a subject. Such compositions typically comprise the compositions of the invention and a pharmaceutically acceptable carrier. Methods for preparing phytochemical compositions formulated with a pharmaceutically acceptable carrier suitable for oral delivering are well known by persons of skill in the art; for example, such methods are described in U.S. Pat. No. 7,241,461, and in U.S. Pat. No. 7,067,159, both of which are incorporated herein by reference in their entirety. The orally administered phytochemical composition or the oral dietary supplement of the invention can be used in beverages, tonics, infusions, or foodstuffs alone, or in combination with other dietary supplements or therapeutics.

The orally administered phytochemical composition or the oral dietary supplement of the invention is administered as a daily dose containing from 50 to 6000 mg of the phytochemical composition to effectively downregulate inflammation in the subject. In one embodiment, the orally administered phytochemical composition or the oral dietary supplement of the invention is administered as a daily dose containing an amount selected from 50-250 mg, 250-500 mg, 500-1000 mg, 1000-1500 mg, 1500-2000 mg, 2000-2500 mg, 2500-3000 mg, 3000-3500 mg, 3500-4000 mg, 4000-4500 mg, 4500-5000 mg, 5000-5500 mg, and 5500-6000 mg of the phytochemical composition to effectively downregulate inflammation in the subject. In another embodiment, the orally administered phytochemical composition or the oral dietary supplement of the invention is administered as a daily dose containing an amount selected from 1000-2000 mg, 1000-3000 mg, 1000-4000 mg, 1000-5000 mg, 1000-6000 mg, 2000-3000 mg, 2000-4000 mg, 2000-5000 mg, 2000-6000 mg, 3000-4000 mg, 3000-5000 mg, 3000-6000 mg, 4000-5000 mg, 4000-6000 mg, and 5000-6000 mg of the phytochemical composition to effectively downregulate inflammation in the subject. In another embodiment, the orally administered phytochemical composition or the oral dietary supplement of the invention is administered as a daily dose containing an amount selected from 50 mg, 100 mg, 150 mg, 200 mg, 250 mg, 500 mg, 1000 mg, 1500 mg, 2000 mg, 2500 mg, 3000 mg, 3500 mg, 4000 mg, 4500 mg, 5000 mg, 5500 mg, and 6000 mg of the phytochemical composition to effectively downregulate inflammation in the subject. It is understood, that a specific dose level for any particular patient will depend upon a variety of factors, including the activity of the specific compound employed; the age, body weight, general health, sex, and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The present invention provides methods and phytochemical compositions for producing dietary supplements comprising spice combinations that effectively and synergistically downregulate inflammation and promote wellness, good health, and fitness in a human or other mammalian subject. An advantage of the immune regulatory synergistic phytochemical blends of the present invention is that they more closely replicate the health benefits and positive physiological and pharmacological immune parameters associated with good dietary phytonutrition without the toxic side-effects so often associated with drugs.

Another advantage of the synergistically-active phytochemical blends of the present invention is that they can enhance the health benefits of physiological diet and pharmacological drug potency to achieve immune regulatory efficacy. Such phytochemical blends can be accomplished as spices and seasoning as part of a healthy diet, dietary supplements or as an adjuvant to drug therapies.

The synergistically-active phytochemical blends of the present invention can improve the homeostatic “balance” of immune regulation of a subject to reduce pathological immunity. Without loss of efficacy, synergistically-active phytochemical blends of the present invention can be tailored to any commercial or cultural taste and can be fortified with single phytochemicals.

Synergistically-active phytochemical blends of the present invention can potentially provide improved healthcare at lower cost. This might be especially beneficial to improve poor diets in developing countries and counter the calorie-rich, nutrient-poor diet in many developed nations.

### EXAMPLES

The invention will be further explained by the following Examples, which are intended to be purely exemplary of the invention, and should not be considered as limiting the invention in any way.

### Example 1

Freshly isolated human PBMCs were incubated in cell culture medium containing an extract of turmeric, ginger, or a combination of turmeric and ginger extracts, at dilutions ranging from 1:30 to 1:93,750, for 6-7 hours. Cells were also incubated in spice-free cell culture medium (negative control) and in medium containing 8.3 ug/ml of curcumin, an inhibitor of inflammatory responses (positive control), for the same time. The cells were then cultured in medium containing lipopolysaccharide (LPS), a pro-inflammatory bacterial product, for 12-15 hours, and the supernatants were harvested and assayed by enzyme-linked immunoassay (ELISA) to measure the content of IL-8, a biomarker for cellular inflammatory response.

Cell cultures: 10-ml samples of sodium heparin-anticoagulated peripheral venous blood collected from healthy volunteers in Vacutainer collection tubes (Becton Dickinson Biosciences Inc., Rutherford, N.J., USA) and diluted 1:1 in sterile phosphate-buffered saline (PBS) followed immediately by isolation of PBMC using Ficoll-Hypaque density centrifugation in a Centra-CL-2 centrifuge (MidAtlantic Diagnostics, Inc., Mount Laurel, N.J. 08054 USA) were used to establish cultures for assessment of immunoregulatory capacity of each spice mixture evaluated in the present study. PBMC were suspended in RPMI-1640 complete cell culture medium (GIBCO/Life Technologies Inc., Carlsbad Calif., USA), supplemented with antibiotics, 10% heat-inactivated fetal calf serum and 1.0×10⁻⁵ M beta-mercaptoethanol (Sigma Immunochemicals, St. Louis Mo., USA).

Incubation with spice solutions: Suspensions of PBMC at 1.5×10⁴ cells/well in sterile 96-well round-bottom microtiter plates (BD Biosciences) were incubated with selected dilutions of liquified extracts of turmeric and ginger, individually and in combinations (Aqua Spice liquified spice extracts, Kancor Flavors and Extracts, LTD, Kerala, India), or with dilutions of purified curcumin (Sigma, St. Louis), for 6-7 hours in a volume of 100 ul per culture at 37° C., 5% CO₂, fully humidified, in quadruplicate for each culture condition. The serial dilutions of the spice extracts were prepared as 2∴ solutions, and the cell cultures were prepared by adding 50 ul of a 2× spice extract dilution to 50 ul of cell suspension in RPMI in a well of a 96-well round bottom plate.

Stimulation of cellular inflammatory responses: The cells were then administered a “primary insult” to activate pro-inflammatory signaling processes, by incubation for from 12 to 15 hours with 4 ng/ml lipopolysaccharide (LPS) in a volume of 150 ul, followed by centrifugation of culture plates using a Damon IEC HN-SII centrifuge (GMI, Inc. Ramsey, Minn., USA), Trypan Blue dye exclusion assays to assess cell viability in each culture, harvest of culture supernatants and estimation of PMBC IL-8 expression using ELISA.

Detection of IL-8 biomarker: Analysis of cell culture supernatants for IL-8 content was conducted using a Sandwich ELISA protocol. Briefly, 96-well, flat-bottom microtiter plates (BD Biosciences, Rutherford, N.J., USA) were coated with mouse anti-human IL-8 polyclonal antibody (Thermo Fisher Scientific, Rockford Ill., USA Product M801), incubated with PBMC culture supernatants for IL-8 capture, then with mouse-anti-human IL-8 biotinylated monoclonal detecting antibody (Thermo Fisher Product M802B), followed by incubation with HRP-conjugated streptavidin (Thermo Fisher Product N100). Next, each ELISA plate was developed with 1-step Ultra TMB ELISA substrate (Thermo Fisher Product 34028) and relative expression of IL-8 under each culture condition was evaluated as absorbance of the developed substrate at 450 nm in a Synergy HT Multi-Mode Micro plate Reader.

Results: To determine the ability of the tested spice preparations to suppress expression of the inflammatory biomarker IL-8, PBMCs were isolated from a healthy donor and cultured in the presence of various spices for 6-7 hours. Cells were then challenged with LPS for 12-15 hours, after which cell culture medium was withdrawn and assayed for IL-8. Using this experimental approach, each spice extract examined in the present study was observed to independently suppress IL-8 production by LPS-stimulated PBMC relative to the negative control (media-treated cultures). Turmeric exhibited particularly high effectiveness in this respect, showing inhibition of LPS-stimulated IL-8 production at dilutions up to 1:18,750 (p<0.05) (FIG. 1A). Inhibition of IL-8 was also observed in the cultures treated with ginger, which inhibited IL-8 production to levels significantly lower than the positive control (curcumin) at a dilution of 1:3,750 (p<0.05) (FIG. 1B).

Synergistic inhibition: IL-8 expression by LPS-stimulated PBMCs was significantly inhibited (by about 50%) by a 1:1 combination of turmeric and ginger (p<0.05) (FIG. 1C) at a dilution of 1:93,750. The degree of inhibition of IL-8 production by the combinations of turmeric and ginger extracts at a dilution of 1:93,750 was significantly greater than the sum of the inhibitory effects of the individual spice extracts at this dilution, and is evidence of synergistic inhibition of LPS-stimulated expression of the IL-8 inflammatory biomarker by combination of turmeric and ginger.

### Example 2

Freshly isolated human PBMCs were incubated in cell culture medium containing an extract of turmeric or capsicum (Aqua Spice liquified spice extracts, Kancor Flavors and Extracts, LTD, Kerala, India), or a 1:1 combination of turmeric and capsicum extracts, at dilutions ranging from 1:30 to 1:93,750, for 6-7 hours. Cells were also incubated in spice-free cell culture medium (negative control), and in medium containing 8.3 ug/ml of curcumin (positive control), for the same time. The cells were then cultured in medium containing LPS for 12-15 hours, and the supernatants were harvested and assayed by ELISA to measure the content of IL-8, a biomarker for cellular inflammatory response, as described in Example 1.

Results: As shown in FIGS. 2A and 2B, treatment of cells with turmeric and capsicum extracts prior to stimulation with LPS effectively suppressed IL-8 production. Turmeric inhibited LPS-stimulated IL-8 production at dilutions up to 1:18,750 (p<0.05) (FIG. 2A), and inhibition of IL-8 was observed in cultures treated with capsicum at a dilution of up to 1:3,750 (p<0.05) (FIG. 2B). Both turmeric and capsicum dilutions inhibited LPS-stimulated IL-8 production to levels significantly lower than the positive control (curcumin). A 1:1 combination of turmeric and capsicum strongly inhibited LPS-stimulated IL-8 production, by about 80%, at a dilution of 1:93,750 (p<0.05) (FIG. 2C), which is significantly greater than the sum of the inhibitory effects of the individual turmeric and capsicum extracts at this dilution. This result demonstrates that the combination of turmeric and capsicum synergistically inhibits LPS-stimulated expression of the IL-8 inflammatory biomarker.

### Example 3

Freshly isolated human PBMCs were incubated in cell culture medium containing an extract of paprika or nutmeg (Aqua Spice liquified spice extracts, Kancor Flavors and Extracts, LTD, Kerala, India), or a 1:1 combination of paprika and nutmeg extracts, at dilutions ranging from 1:30 to 1:93,750, for 6-7 hours. Cells were also incubated in spice-free cell culture medium (negative control) and in medium containing 8.3 ug/ml of curcumin (positive control) for the same time. The cells were then cultured in medium containing LPS for 12-15 hours, and the supernatants were harvested and assayed by ELISA to measure the content of IL-8, a biomarker for cellular inflammatory response, as described in Example 1.

Results: As shown in FIGS. 3A and 3B, treatment of cells with paprika and nutmeg extracts prior to stimulation with LPS effectively suppressed IL-8 production. Paprika inhibited LPS-stimulated IL-8 production at dilutions of 1:30 and 1:150, but not at 1:750 or higher dilution (p<0.05) (FIG. 3A). Nutmeg significantly inhibited LPS-stimulated IL-8 production (>50%) at dilutions of 1:30, 1:150, and 1:750, while giving a low level of inhibition (≦about 20%) at dilutions of 1:750, 1:18,750, and 1:93,750 (p<0.05) (FIG. 3B). A 1:1 combination of paprika and nutmeg significantly inhibited LPS-stimulated IL-8 production (by about 50%) at a dilution of 1:3,750 (p<0.05) (FIG. 3C), which is significantly greater than the sum of the inhibitory effects of the individual paprika and nutmeg extracts at this dilution. This result demonstrates that the combination of paprika and nutmeg also synergistically inhibits LPS-stimulated expression of the IL-8 inflammatory biomarker.

### REFERENCES

Aftab N, Vieira A. 2009. Antioxidant activities of curcumin and combinations of this curcuminoid with other phytochemicals. Phytother Res Nov 19.

Aggarwal B B, Kumar A, Bharti A C. 2003. Anticancer potential of curcumin: preclinical and clinical studies. Anticancer Res 23(1A):363-398.

Aggarwal B B, Shishodia S. 2004. Suppression of the nuclear factor-kappaB activation pathway by spice-derived phytochemicals: reasoning for seasoning. Ann N Y Acad Sci 1030: 434-41.

Ashburner B P, Westerheide S D, Baldwin A S Jr. 2001. The p65 (RelA) subunit of NF-kappaB interacts with the histone deacetylase (HDAC) corepressors HDAC1 and HDAC2 to negatively regulate gene expression. Mol Cell Biol. 20:7065-7077.

Attaran D, Lari S M, Khajehdaluee M, Ayatollahi H, Towhidi M, Asnaashari A, Marallu H G, Mazloomi M, Mood M B. Highly sensitive C-reactive protein levels in Iranian patients with pulmonary complication of sulfur mustard poisoning and its correlation with severity of airway diseases. Hum Exp Toxicol. 2009 28(12):739-45.

Bush G W. 2007 Homeland Security Presidential Directive/HSPD-18. Medical Countermeasures against Weapons of Mass Destruction.

Cowan F M, Broomfield C A, and Smith W J. 2002. Suppression of sulfur mustard-increased IL-8 in human keratinocyte cell cultures by serine protease inhibitors: Implications for toxicity and medical countermeasures. Cell Biology and Toxicology 18:175-180.

Cowan F M, Broomfield C A, Nealley E W, Petrali J P. 2006. Anti-Inflammatory and Morphocytotoxic Actions of 4-Methyl-2-Mercaptopyridine-1-oxide in Sulfur Mustard-Exposed Skin and Lung Epithelial Cell Cultures: Implications for Broad Spectrum Countermeasures. US Army Medical Research and Materiel Command Medical defense Bioscience Review, Baltimore Md.

Cowan F M, Broomfield C A, Stojiljkovic M P and Smith W J. 2004 A Review of Multi-Threat Medical Countermeasures Against Chemical Warfare and Terrorism. Military Medicine 169: 850-855.

Chung H Y, Kim H J, Kim K W, Choi J S, Yu B P. 2002. Molecular inflammation hypothesis of aging based on the anti-aging mechanism of calorie restriction. Microsc Res Tech 59(4): 264-72.

Das D K, Mukherjee S, Ray D. 2010. Resveratrol and red wine, healthy heart and longevity. Heart Fail Rev. Mar. 18, 2010. [Epub ahead of print]

Dhillon N, Aggarwal B B, Newman R A, Wolff R A, Kunnumakkara A B, Abbruzzese J L, Ng C S, Badmaev V, Kurzrock R. 2008 Phase II trial of curcumin in patients with advanced pancreatic cancer. Clin Cancer Res 14(14): 4491-4499.

Eggesbo J B, Hjermann I, Lund P K, Joo G B, Ovstebo R, Kierulf P. LPS-induced release of IL-1 beta, IL-6, IL-8, TNF-alpha and sCD14 in whole blood and PBMC from persons with high or low levels of HDL-lipoprotein. Cytokine. 1994 Sep; 6(5):521-9.

Gerber, L. M., G. C. Williams, and S. H. Gray. 1999. The nutrient-toxin dosage continuum in human evolution and modern health. Q. Rev. Biol. 74:273-289.

Giardina, A, Hosseini, A, Haines, D. Chronic Illness in Toxicant-Exposed Populations. 2009. Charles University of Connecticut Department of Molecular and Cell Biology.

Haines D, Varga B, Bak I, Juhasz B, Mahmoud F, Kalantari H, Gesztelyi R, Varga E, Tosaki A. Summative Interaction Between Astaxanthin, Ginkgo biloba Extract (EGb761) and Vitamin C in Suppression of Respiratory Inflammation: A Comparison with Ibuprofen Phytoth Res 2010, In print.

Haines D D, Bak I, Ferdinandy P, Mahmoud F F, Al-Harbi S A, Blasig I E, Tosaki A. 2000. Cardioprotective effects of the calcineurin inhibitor FK506 and the PAF receptor antagonist and free radical scavenger, EGb 761, in isolated ischemic/reperfused rat hearts. J Cardiovasc Pharmacol 35: 37-44.

Kawahara T L, Michishita E, Adler A S, Damian M, Berber E, Lin M, McCord R A, Ongaigui K C, Boxer L D, Chang H Y, Chua K F. 2009. SIRT6 links histone H3 lysine 9 deacetylation to NF-kappaB-dependent gene expression and organismal life span. Cell. 2009136(1): 62-74.

Liu R H. 2004. Supplement: International Research Conference on Food, Nutrition, and Cancer Potential Synergy of Phytochemicals in Cancer Prevention: Mechanism of Action. The American Society for Nutritional Sciences J Nutr 134:3479S-3485S.

Mahmoud Abul H T, Haines D D, Abal A T, Wise J A. In vitro effects of ginkgo lide B combined with cyclosporin A on T-lymphocyte activation and IL-5 expression in peripheral blood mononuclear cells from asthmatic subjects. Transplant Proc. 2002 Nov;34(7):2958-61.

Martins, K R. 2006. Targeting apoptosis with dietary bioactive agents. Exp Biol Med 231: 117-129.

McCarty M F. 2004. Targeting multiple signaling pathways as a strategy for managing prostate cancer: multifocal signal modulation therapy. Integrative Cancer Therapies 3(4); pp. 349-380.

Myzak M C, Tong P, Dashwood W-M, Dashwood R H, Ho E. 2007. Sulforaphane retards the growth of human PC-3 xenografts and inhibits HDAC activity in human subjects. Exp Biol Med 232(2): 227-234.

Morré, D J, M. Morré, D M. 2003. Synergistic Capsicum—tea mixtures with anticancer activity. JPP 55: 987-994

Oyagbemi et al. 2010. Capsaicin: a novel chemopreventive molecule and its underlying molecular mechanisms of action. Indian J. Cancer. 47(1): 53-8.

Pourfarzam S, Ghazanfari T, Yaraee R, Ghasemi H, Hassan Z M, Faghihzadeh S, Ardestani S K, Kariminia A, Fallahi F, Soroush M R, Merasizadeh J, Mahlojirad M, Naghizadeh M M, Ghanei M. Serum levels of IL-8 and IL-6 in the long term pulmonary complications induced by sulfur mustard: Sardasht-Iran Cohort Study. Int Immunopharmacol. 2009 ;9(13-14):1482-8.

Surbatovic M, Filipovic N, Radakovic S, Stankovic N, Slavkovic Z. 2007. Immune cytokine response in combat casualties: blast or explosive trauma with or without secondary sepsis. Mil Med 172: 190-195.

Underwood, “Quieting a Body's Defenses,” Newsweek, Jun. 10, 2005. Vadivelu et al. 2010. Recent advances in postoperative pain management. Yale J. Biol. Med., 83: 11-25.

Yeung F, Hoberg J E, Ramsey C S, Keller M D, Jones D R, Frye R A Mayo M W. 2004. Modulation of NF-B-dependent transcription and cell survival by the SIRT1 deacetylase. EMBO Journal 23: 2369-2380.

1. A phytochemical composition formulated for oral administration comprising a mixture of two or more phytochemical constituents selected from the group consisting of a spice, a partially purified phytochemical, and a fully purified phytochemical,
wherein two or more of the phytochemical constituents are present in amounts effective to downregulate inflammation in a mammal following oral administration of the phytochemical composition to the mammal.

2. The phytochemical composition of claim 1, wherein the biomarker is selected from the group consisting of C-reactive protein (CRP), interleukin (IL)-1b, IL-6, IL-8, IL-10, tumor necrosis factor-alpha (TNF-alpha), granulocyte macrophage-colony stimulating factor (GM-CSF), macrophage inflammatory protein 1 alpha (MIP-1a), RANTES, prostaglandin E2 (PGE2), nuclear factor-kappa B (NF-kappaB), a heat shock protein (hsp), histone deacetylase (HDAC), intercellular adhesion molecule 1 (ICAM 1), myeloperoxidase (MPO), and soluble CD40 ligand (sCD40L).

3. The phytochemical composition of claim 1, wherein the phytochemical composition comprises two spices selected from the group consisting of (a) turmeric and ginger, (b) turmeric and capsicum, and (c) paprika and nutmeg, in amounts effective to downregulate inflammation in a mammal following oral administration of the phytochemical composition.

4. The phytochemical composition of claim 3, comprising 5% to 70% turmeric and 3% to 50% ginger.

5. The phytochemical composition of claim 3, comprising 5% to 70% turmeric and 0.05% to 30% capsicum.

6. The phytochemical composition of claim 3, comprising 5% to 70% turmeric, 3% to 50% ginger, 0.5% to 50% cinnamon, and 0.05% to 30% capsicum.

7. The phytochemical composition of claim 6, which further comprises 0.5% to 15% grape seed extract, and 0.5% to 15% grape skin extract.

8. The phytochemical composition of claim 6, wherein at least one spice in the phytochemical composition selected from turmeric, ginger, cinnamon, and capsicum is a spice extract.

9. The phytochemical composition of claim 8, further comprising at least one emulsifier that increases the aqueous solubility of at least one extract in the phytochemical composition selected from turmeric extract, ginger extract, cinnamon extract, and capsicum extract.

10. The phytochemical composition of claim 3, formulated for oral administration to a human of a daily dosage of 50 to 6000 mg of the phytochemical composition.

11. A phytochemical composition comprising 5% to 70% turmeric extract, 3% to 50% ginger extract, 0.5% to 50% cinnamon extract, and 0.05% to 30% capsicum extract.

12. The phytochemical composition of claim 11, which further comprises 0.5% to 15% grape seed extract, and 0.5% to 15% grape skin extract.

13. The phytochemical composition of claim 11, further comprising at least one emulsifier that increases the aqueous solubility of at least one extract in the phytochemical composition selected from turmeric extract, ginger extract, cinnamon extract, and capsicum extract.

14. The phytochemical composition of claim 11, formulated for oral administration to a human of a daily dosage of 50 to 6000 mg of the phytochemical composition.

15. A phytochemical composition comprising 10% to 50% turmeric, 5% to 30% ginger, 1% to 40% cinnamon, and 0.1% to 5% capsicum.

16. The phytochemical composition of claim 15, wherein at least one spice selected from turmeric, ginger, cinnamon, and capsicum is a spice extract comprising at least one emulsifier that increases its aqueous solubility.

17. The phytochemical composition of claim 15, which further comprises 1% to 10% grape seed extract, and 1% to 10% grape skin extract.

18. The phytochemical composition of claim 15, formulated for oral administration to a human of a daily dosage of 50 to 6000 mg of the phytochemical composition.

19. A dietary supplement consisting essentially of 10% to 50% turmeric extract, 5% to 30% ginger extract, 1% to 40% cinnamon extract, and 0.1% to 5% capsicum extract, and at least one emulsifier that increases the aqueous solubility of one or more of the turmeric, ginger, cinnamon, and capsicum extracts.

20. The dietary supplement of claim 19, formulated for oral administration to a human of a daily dosage of 50 to 6000 mg of the dietary supplement.

21. A method for making a dietary supplement comprising a phytochemical composition that downregulates inflammation in a mammal, said method comprising preparing a dietary supplement comprising the phytochemical composition of claim 1.

22. A method for alleviating or reducing undesirable effects of inflammation in a mammalian subject, said method comprising orally administering to the subject the phytochemical composition of claim 11.

23. A method for preventing undesirable effects of inflammation in a mammalian subject, said method comprising orally administering to the subject the phytochemical composition of claim 11.

24. A process for manufacturing an orally administered phytochemical composition for reducing or preventing undesirable effects of inflammation in a subject, said method comprising admixing phytochemical preparations to yield an admixture that effectively downregulates inflammation and inflammatory response in the subject, wherein the admixture comprises the phytochemical composition of claim 17.
